(19) United States
(12) Patent Application Publication
(10) Pub. No.: US 2026/0240165 A1
(43) Pub. Date: Aug. 20, 2026

(54) NITRIC OXIDE-RELEASING COMPOSITIONS AND APPLICATIONS THEREOF

(71) Applicant: University of Georgia Research Foundation, Inc., Athens, GA (US)

(72) Inventors: Elizabeth J. Brisbois, Athens, GA (US); Hitesh HANDA, Athens, GA (US); Arnab MONDAL, Loganville, GA (US); Grace NGUYEN, Athens, GA (US)

(21) Appl. No.: 19/640,604

(22) Filed: Apr. 7, 2026

Related U.S. Application Data
(63) Continuation-in-part of application No. PCT/US2024/050337, filed on Oct. 8, 2024.
(60) Provisional application No. 63/588,794, filed on Oct. 9, 2023.

(51) Int. Cl. A01N 59/00; A01N 25/04; A01N 25/10; A01N 25/18; A01N 25/30

(52) U.S. Cl. CPC A01N 59/00; A01N 25/04; A01N 25/10; A01N 25/18; A01N 25/30

(57) ABSTRACT

Described herein are compositions comprising a nitric oxide-releasing material comprising a (i) a polysiloxane network and (ii) a plurality of nitric oxide-donating moieties covalently bonded to the polysiloxane network; and (b) a nanonemulsion comprising a surfactant and a hydrophobic solvent. Also described herein are articles composed of the compositions described herein and methods for using the same. The compositions described herein provide highly sustained, long-term nitric oxide release that have numerous biological activities such as, for example, preventing localized platelet activation, fibrinogen adhesion, and biofilm formation for a prolonged period of time.

### CROSS-REFERENCE TO RELATED APPLICATIONS

This application is a continuation in-part of International Application No. PCT/US2024/050337, filed on Oct. 8, 2024, which claims the benefit of and priority to U.S. Provisional Patent Application No. 63/588,794, filed on Oct. 9, 2023, the contents of which are incorporated by reference herein in their entireties.

### STATEMENT REGARDING FEDERALLY SPONSORED RESEARCH OR DEVELOPMENT

This invention was made with government support under award R01HL151473 awarded by the National Institutes of Health. The government has certain rights in the invention.

### BACKGROUND

While many hospital procedures are not invasive, some, like surgery or device implantation, require absolute care and sterility to prevent adverse events from occurring. However, according to the Centers for Disease Control and Prevention (CDC), about 2 million hospital-associated infections (HAIs) are reported, and 50-70% of the reported infections are caused by indwelling medical devices.^{1,3} Central line-associated bloodstream infections (CLABSIs), a category of catheter infections, contribute to about 60% of all HAIs in vascular access devices.^{3,4} These infections could lead to biofilm formation, when planktonic (free-floating) bacteria adhere to a foreign surface, proliferate, secrete extracellular protein substances (EPS) for protection, and disperse to proliferate at other locations.⁵ Biofilms are even more difficult to penetrate and handle than a normal infection using traditional antibiotics; studies have shown that concentrations one thousand times the minimum inhibitory concentration (MIC) still does not effectively reduce the biofilm viability.^{6,7} Once the biofilms are mature, they can migrate to other locations along the material surface or within the body to cause other infections.⁸ These infections could lead to worse conditions, such as sepsis, the body's extreme reaction/inflammation to infection, and/or death. Another major complication of implanted medical devices is thrombosis or blood clots; in extreme cases, the clots can break apart and travel through the bloodstream to a different location in the body (e.g., lungs—pulmonary embolism) and cause another clot and prevent proper device function.⁹ Although preventable and reversible via systemic administration of anticoagulants, such as heparin, warfarin, and argatroban,¹⁰⁻¹² these incidences can be deadly. About 2 million Americans are affected by thrombosis annually; at least 100,000 and up to 300,000 cases are fatal.¹³ Not only is the death toll high, but treatment for clotting can also cost up to $20,000 per case with an estimated total cost of up to $14 billion per year.¹³ Current gold standards for treating infections and thrombosis include systemic antibiotic¹⁴ and anticoagulant (i.e., oral anticoagulant)¹⁵ administration; however, systemic administration of these drugs could cause adverse effects like antimicrobial resistance, internal bleeding, more bruising, and excessive bleeding, which should be avoided because these could lead to death.^{15,16}

### SUMMARY

Described herein are compositions comprising a nitric oxide-releasing material comprising a (i) a polysiloxane network and (ii) a plurality of nitric oxide-donating moieties covalently bonded to the polysiloxane network; and (b) a nanonemulsion comprising a surfactant and a hydrophobic solvent. Also described herein are articles composed of the compositions described herein and methods for using the same. The compositions described herein provide highly sustained, long-term nitric oxide release that have numerous biological activities such as, for example, preventing localized platelet activation, fibrinogen adhesion, and biofilm formation for a prolonged period of time.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows NO-releasing, NE-infused antibacterial and slippery surface. The bulk substrate, covalently bound SNAP-PDMS, provides prolonged NO release and effective antibacterial properties, while the n-hexadecane-based NE promotes anti-fouling properties by preventing bacteria and platelet adhesion.

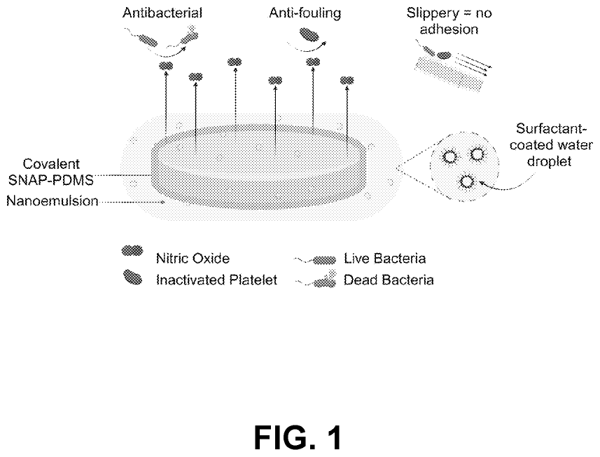

FIG. 2A shows dynamic light scattering (DLS) was performed by diluting the NE in a 1:200 ratio with n-hexadecane; the diameters of the water droplets in the dilution were then measured using a Zetasizer Nano ZS. The measurements showed that the droplet particles of ~150 nm had the greatest distribution in the total volume of the NE. FIGS. 2B and 2C show (B) swelling ratio and (C) swelling rate of the control PDMS and covalent SNAP-PDMS. First, the weights of the base polymers were measured. Then, the samples were immersed in the NE; the weight was measured at different time points, and the swelling ratio and rates were calculated according to Equations 1 and 2. The polymers did not significantly swell after ~4 h, demonstrated by the stabilization of the swelling ratio and rate after 4 h (n=5, p<0.05).

FIG. 3A shows the mechanism of fouling material sliding off NE-swelled surfaces. The surface NE encapsulates the fouling material and initiates a sliding action to repel the fouling agent. FIG. 3B shows the initial water sliding angle was measured by measuring the first angle at which a water droplet slides off the surface when tilted. The angles show the initial slippery nature of the NE-swelled polymers with a sliding angle below 20°, while the non-NE-swelled polymers report sliding angles of 90° because the water droplets were pinned to the surface (n 3, (***) p<0.001). FIG. 3C shows the water sliding angle of slippery samples over 7 days in physiological conditions, where the samples were immersed in 10 mM PBS w/100 μM EDTA at 37° C. and shaking conditions between time points, remains below 20°, indicating the self-healing ability of the NE lubricant layer from the lubricant reservoir (n≥3, (*) p<0.05, (***) p<0.001).

FIG. 4A shows the NO release profile of SNAP-PDMS and SNAP-PDMS-NE as measured by chemiluminescence detection under physiological conditions. As NO release is catalyzed in the presence of heat and moisture, the NO-releasing samples were immersed in 10 mM PBS w/100 μM EDTA at 37° C. in the sample chamber, and the NO released was quantified. The slightly higher release seen on Day 0 of the SNAP-PDMS-NE samples allude to the polymer matrix itself swelling to accommodate the NE infused, thereby allowing the S—NO bond to readily release NO (n=4, p>0.05). FIG. 4B shows the cumulative NO released from SNAP-PDMS and SNAP-PDMS-NE over 7 days.

FIG. 5A shows the biological assessment using a bacterial adhesion assay over 24 h against S. aureus. Samples were incubated in bacterial solution for 24 h. Adhered bacteria were removed from the surface via homogenization and quantified. SNAP-PDMS-NE showed 3.5 log reduction of adhered bacteria compared to control (n=6, (***) p<0.001, (****) p<0.0001). FIG. 5B shows the biological assessment using a bacterial adhesion assay over 24 h against E. coli. Samples were incubated in bacterial solution for 24 h. Adhered bacteria were removed from the surface via homogenization and quantified. SNAP-PDMS-NE showed 1.5 log reduction of adhered bacteria compared to control (n=5, (**) p<0.01, (***) p<0.001, (****) p<0.0001).

FIGS. 6A-6C show the biological assessments for SNAP-PDMS-NE against controls. (A) Relative cell viability of 3T3 mouse fibroblast cells was measured via MTT assay. Samples leachates were exposed to the cells, and the MTT assay quantified the live cell metabolic activity. Cytocompatibility assessments indicated that all sample types showed a relative cell viability >70% (n=4, p>0.05). (B) Fibrinogen adsorbed to the surface. The samples were pre-incubated with CMF-PBS to reach surface saturation prior to incubation with FITC-Fg. Expectedly, the SNAP-PDMS demonstrated higher amounts of Fg adsorbed as the protein tends to adsorb more to NO-releasing surfaces. SNAP-PDMS-NE showed a reduction of Fg adsorbed, asserting that the presence of the slippery surface aided in preventing protein adsorption (n=8, (*) p<0.05, (**) p<0.01). (C) Adhered platelets were quantified on sample surfaces after incubation in platelet rich plasma for 1.5 h. Lactate dehydrogenase (LDH) assay showed a significant reduction of platelet adhesion of the NE-swelled samples compared to the non-NE-swelled samples, indicating that the slippery surfaces significantly contribute to the anti-fouling ability (n=8, (*) p<0.05, (***) p=0.0001, (****) p<0.0001).

FIG. 7A shows the synthesis of NAPTH via thiolactone ring formation on N-acetylpenicillamine. The reaction entails the conversion of acetic anhydride to acetic acid in the presence of anhydrous pyridine. The thiol ring formation acts as a stabilization for the NAP during the conjugation process to hydroxy-terminated PDMS. The resulting NAPTH crystals were triple-washed with equal parts hydrochloric acid, precipitated with hexanes (mix of isomers), and used to make SNAP-PDMS. FIG. 7A shows the synthesis of SNAP-PDMS. The conjugation of NAPTH to hydroxy-terminated PDMS and subsequent nitrosation of NAP-PDMS via tert-butyl nitrite creates a stable NO-releasing polymer (SNAP-PDMS) that is resistant to SNAP leaching. FIG. 7C shows the NO release mechanism of SNAP-PDMS. As SNAP is a RSNO, the NO release can be catalyzed by heat, light, and metal ions; 2 molecules of SNAP-PDMS releases 2 molecules of NO while dimerizing to form a disulfide bond with the residual molecules.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” “having”, “has,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a polysiloxane” includes, but is not limited to, mixtures or combinations of two or more such polysiloxanes, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. The term “alkyl” also refers to alklylene groups represented by the general formula —(CHR)ₙ—, where R is an alky group as defined above (e.g., methyl, ethyl, etc.) and n is an integer from 1 to 20. Examples of alklylene groups include, but are not limited to, methylene, ethylene, propylene, and the like.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g. have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, having from one to ten carbons, or from one to six carbon atoms in its backbone structure. Likewise, “lower alkenyl” and “lower alkynyl” have similar chain lengths. In embodiments described in the present application, preferred alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S— alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O— alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

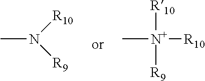

wherein R₉, R₁₀, and R′₁₀ each independently represent a hydrogen, an alkyl, an alkenyl, —(CH₂)ₘ—R₃ or R₉ and R₁₀ taken together with the N atom to which they are attached complete a heterocycle having from 4 to 8 atoms in the ring structure; R₃ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkyl, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

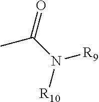

wherein R₉ and R₁₀ are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or non-aromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, and preferably from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

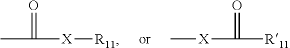

wherein X is a bond or represents an oxygen or a sulfur, and R₁₁ represents a hydrogen, an alkyl, a cycloalkyl, an alkenyl, an cycloalkenyl, or an alkynyl, R′₁₁ represents a hydrogen, an alkyl, a cycloalkyl, an alkenyl, an cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms include, but are not limited to boron, nitrogen, oxygen, phosphorus, sulfur and selenium. Other heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms (for example, 1-14 carbon atoms), and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various aspects, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, such as random, block, graft, etc. The copolymers can have any end-group, including capped or acid end groups.

The term “prevent” or “preventing” as used herein is defined as eliminating or reducing the likelihood of the occurrence of one or more symptoms of a disease or disorder (e.g., biofilm formation) when using the compositions as described herein when compared to a control where the composition is not used.

### Nitric Oxide Releasing Materials and Articles and Methods of Making and Uses Thereof

The need for more biocompatible materials for medical devices remains a major concern because infection, thrombosis, and biofouling have become some of the leading causes of medical device failure and patient morbidity and mortality. Described herein are materials that combine the antibacterial capabilities of a nitric oxide (NO)-releasing substrate with the anti-fouling effects of a slippery surface produced with the use of a nanoemulsion (NE).

Described herein are nitric oxide-releasing compositions. In one aspect, the compositions comprise a nitric oxide-releasing material comprising a (i) a polysiloxane network and (ii) a plurality of nitric oxide-donating moieties covalently bonded to the polysiloxane network; and (b) a nanonemulsion comprising a surfactant and a hydrophobic solvent. The compositions described herein provide highly sustained, long-term nitric oxide release that have numerous biological activities.

In one aspect, the polysiloxane network in the nitric oxide-releasing material is the reaction product between polysiloxane and an amine-functionalized crosslinker. In one aspect, the polysiloxane comprises one or more functional groups that can react with the amine-functionalized crosslinker. In one aspect, the polysiloxane includes two or more hydroxyl and/or amino groups. In another aspect, the polysiloxane is terminated with a hydroxyl group, which is referred to herein as a “hydroxy-terminated polysiloxane.” For example, if the polysiloxane is linear, then each end of the polysiloxane is terminated with a hydroxyl group. In other aspects, when the polysiloxane is branched, then each branch of the polysiloxane is terminated with a hydroxyl group.

In one aspect, the polysiloxane is a polydimethylsiloxane, a polydiethylsiloxane, a polydipropylsiloxane, or a polydiphenylsiloxane. In another aspect, the polysiloxane used to make the polysiloxane network is a polydimethylsiloxane, a polydiethylsiloxane, a polydipropylsiloxane, or a polydiphenylsiloxane terminated with a hydroxyl group. In one aspect, the polysiloxane has a kinematic viscosity of about 2,500 cSt to about 4000 cSt, or about 2,500 cSt, 2,550 cSt, 2,600 cSt, 2,650 cSt, 2,700 cSt, 2,750 cSt, 2,800 cSt, 2,850 cSt, 2,900 cSt, 2,950 cSt, 3,050 cSt, 3,100 cSt, 3,150 cSt, 3,200 cSt, 3,250 cSt, 3,300 cSt, 3,350 cSt, 3,400 cSt, 3,450 cSt, 3,500 cSt, 3,550 cSt, 3,600 cSt, 3,650 cSt, 3,700 cSt, 3,750 cSt, 3,800 cSt, 3,850 cSt, 3,900 cSt, 3,950 cSt, or 4,000 cSt, where any value can be a lower and upper endpoint of range (e.g., 2,550 cSt to 3,600 cSt).

In one aspect, the amine-functionalized crosslinker includes one or more groups that react with the polysiloxane to form the polysiloxane network. In one aspect, amine-functionalized crosslinker is an amino silane compound. In one aspect, when the polysiloxane is terminated with hydroxyl groups, the hydroxyl group reacts with the silane group of the amino silane compound. In one aspect, the amino silane compound has the structure:

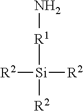

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ heteroalkenyl, a substituted or unsubstituted C₁-C₂₀ alkoxy, or a substituted or unsubstituted C₁-C₂₀ heteroalkoxy; where each occurrence of R² is hydroxy or alkoxy. In one aspect, R¹ is a C₁-C₁₀ alkyl group such as, for example, methylene, ethylene, propylene, butylene, and the like.

A plurality of nitric oxide-donating moieties is covalently bonded to the polysiloxane network. Not wishing to be bound by theory, the polysiloxane network includes a plurality of amino groups derived from the amine-functionalized crosslinker. The amino groups can further react with additional compounds that covalently bond nitric oxide-donating moieties or precursors thereof to produce the polysiloxane network. In one aspect, a compound possessing one or more nitric oxide groups can be reacted directly with the polysiloxane network to produce the nitric oxide releasing material.

In other aspects, the polysiloxane network can be reacted with a compound that possesses one or more groups that are a precursor to the nitric oxide-releasing material. In one aspect, the compound possesses one or more sulfur groups that can be subsequently nitrosylated. In one aspect, the polysiloxane network is reacted with a thiolactone. Not wishing to be bound by theory, the amino groups present in the polysiloxane network react with the thiolactone, where the thiolactone ring-opens to produce a free thiol group or ion. In one aspect. In one aspect, the thiolactone has the structure:

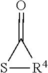

where R⁴ is a substituted or unsubstituted C₁-C₁₂ alkyl (e.g., methylene, ethylene, propylene, butylene).

In another aspect, the thiolactone has the structure:

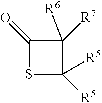

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

In one aspect, the thiolactone is N-acetylcysteine thiolactone, N-acetyl-homocysteine thiolactone, homocysteine thiolactone, butyryl-homocysteine thiolactone, or any combination thereof.

In one aspect, the nitric oxide-releasing material includes a plurality of —S—NO groups. For example, when the polysiloxane network is reacted with a thiolactone as provided above, a plurality of thiol groups is produced. The thiol groups can subsequently be nitrosylated by reacting the free thiol groups with a nitrosylating agent. In one aspect, the nitrosylating agent is t-butyl nitrite, isopentyl nitrite, isobutyl nitrite, amyl nitrite, or cyclohexyl nitrite

In certain aspects, nitrosylation of the thiol group can be performed in the presence of an acid catalyst. In one aspect, the organic acid is an organic sulfonic acid having the formula RS(O)₂OH, where R is an alkyl group or aryl group as defined herein. In one aspect, R is an aryl group substituted with a C₁-C₂₀ alkyl group. In one aspect, the sulfonic acid includes, but is not limited to, dodecylbenzene sulfonic acid, dinonylnaphthalenedisulfonic acid, or 4-octylbenzenesulfonic acid. In other aspects, the organic acid can be acetic acid, formic acid, or lactic acid. In one aspect, the organic acid used is from about 0.1 weight percent to about 2 weight percent of the polysiloxane network, or about 0.1 weight percent, 0.1 weight percent, 0.2 weight percent, 0.3 weight percent, 0.4 weight percent, 0.5 weight percent, 0.6 weight percent, 0.7 weight percent, 0.8 weight percent, 0.9 weight percent, 1.0 weight percent, 1.1 weight percent, 1.2 weight percent, 1.3 weight percent, 1.4 weight percent, 1.5 weight percent, 1.6 weight percent, 1.7 weight percent, 1.8 weight percent, 1.9 weight percent, or 2.0 weight percent, where any value can be a lower and upper endpoint of range (e.g., 0.8 weight percent to 1.2 weight percent).

The nitric oxide-donating moieties covalently bonded to the polysiloxane network can vary depending upon the selection of starting materials used to produce the nitric oxide releasing material. In one aspect, the nitric oxide-donating moiety is a S-nitrosothiol. In another aspect, the S-nitrosothiol is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof. In another aspect, the nitric oxide-donating moiety is a diazeniumdiolate. In one aspect, the diazeniumdiolate is diazeniumdiolated dibutylhexanediamine or a derivative thereof.

In one aspect, the nitric oxide-donating moiety has a structure according to the following structure:

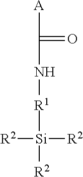

where A is a nitric oxide donor; where R¹ is selected from a substituted or unsubstituted C₁-C₂₀alkyl, a substituted or unsubstituted C₁-C₂₀ heteroalkyl, a substituted or unsubstituted C₂-C₂₀alkenyl, a substituted or unsubstituted C₂-C₂₀ heteroalkenyl, a substituted or unsubstituted C₁-C₂₀ alkoxy, or a substituted or unsubstituted C₁-C₂₀ heteroalkoxy; where each occurrence of R² is independently a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ heteroalkenyl, a substituted or unsubstituted C₁-C₂₀ alkoxy, a substituted or unsubstituted C₁-C₂₀ heteroalkoxy, or a bond to a polysiloxane in the plurality of polysiloxanes wherein at least two occurrences of R² are a bond to the polysiloxane network via a siloxane bond (e.g., —O—Si—O—). In one aspect, A is a S-nitrosothiol group. In another aspect, A has the structure below

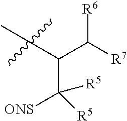

where R⁵, R⁶, and R⁷ are as defined above.

In one aspect, the nitric oxide releasing material can have the structure below:

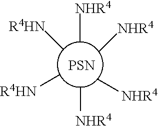

where PSN is the polysiloxane network and R⁴ is a nitric oxide-donating moiety. In one aspect, R⁴ is a S-nitrosothiol group. In another aspect, R⁴ is a residue S-nitroso-N-acetyl-penicillamine, S-nitroso-N-acetyl cysteine, S-nitroso-N-acetyl cysteamine, S-nitrosoglutathione, methyl S-nitrosothioglycolate, or a derivative thereof. In this aspect, the plurality of amino groups derived from the amine-functionalized crosslinker are covalently bonded to the nitric oxide-donating moiety.

The amount of the nitric oxide-donating moieties present in the nitric oxide-releasing material can vary. In one aspect, the nitric oxide-donating moieties are present in an amount from about 0.15 micromoles per milligram of the polysiloxane network to about 0.80 micromoles per milligram of the polysiloxane network, or about 0.15 micromoles per milligram, 0.20 micromoles per milligram, 0.25 micromoles per milligram, 0.30 micromoles per milligram, 0.35 micromoles per milligram, 0.40 micromoles per milligram, 0.45 micromoles per milligram, 0.50 micromoles per milligram, 0.55 micromoles per milligram, 0.60 micromoles per milligram, 0.65 micromoles per milligram, 0.70 micromoles per milligram, 0.75 micromoles per milligram, or 0.80 micromoles per milligram, where any value can be a lower and upper endpoint of range (e.g., 0.30 micromoles per milligram to 0.70 micromoles per milligram). The Examples provide non-limiting methods for producing the nitric oxide releasing materials described herein.

The compositions described herein include a nanoemulsion. In one aspect, the nanoemulsion is a water-in-oil nanoemulsion, which is a mixture that involves at least two immiscible fluids that are forcibly combined through external forces. Here, the oil component is the bulk/continuous phase and water is the dispersed phase. In this aspect, one more surfactants are used to stabilize the nanoemulsion. Nanoemulsions are characterized by their translucent appearance and look like a true solution; however, the emulsions have dispersed droplets whose sizes are in the range of about 10 nm to about 100 nm.

The nanoemulsion can be prepared by mixing a surfactant and a hydrophobic solvent to produce a first mixture followed by adding water to the first mixture with subsequent mixing. In one aspect, the weight ratio of the hydrophobic solvent to surfactant is from 1:1 to 3:1, or 1:1, 1.5:1, 2.0:1, 2.5:1, or 3:1, where any value can be a lower and upper endpoint of range (e.g., 1.5:1 to 2.5:1). In another aspect, the amount of water added to the first mixture of hydrophobic solvent and surfactant is from 1% v/v to 20% v/v, or 1% v/v, 3% v/v, 5% v/v, 7% v/v, 9% v/v, 11% v/v, 13% v/v, 15% v/v, 17% v/v, 19% v/v, or 20% v/v, where any value can be a lower and upper endpoint of range (e.g., 3% v/v to 7% v/v). The first mixture of hydrophobic solvent and surfactant with water can be mixed by techniques know in the art such as, for example, vortexing. The Examples provide non-limiting methods for producing the nanoemulsions described herein.

In one aspect, the surfactant is a nonionic surfactant. In one aspect, the nonionic surfactant can be an alkoxylated nonionic surfactant such as, for example, ethoxylated and propoxylated nonionic surfactants. Alkoxylated surfactants can be selected from the classes of the nonionic condensates of alkyl phenols, nonionic ethoxylated alcohols, nonionic ethoxylated/propoxylated fatty alcohols, nonionic ethoxylate/propoxylate condensates with propylene glycol, and the nonionic ethoxylate condensation products with propylene oxide/ethylene diamine adducts. In another aspect, the nonionic surfactant includes polyoxyethylene (20) sorbitan monolaurate, sorbitan monolaurate, polyoxyethylene 2-lauryl ether, polyoxyethylene 4-lauryl ether, polyglycerol polyricinoleate, or any combination thereof.

In one aspect, the surfactant includes two different nonionic surfactants. In one aspect, the surfactant includes polyoxyethylene (20) sorbitan monooleate and sorbitan monooleate. In another aspect, the surfactant includes polyoxyethylene (20) sorbitan monooleate and sorbitan monooleate at a weight ratio of from 2:1 to 4:1, or 2:1, 2.5:1, 3:1, 3.5:1, or 4:1, where any value can be a lower and upper endpoint of range (e.g., 2.5:1 to 3.5:1).

In one aspect, the hydrophobic solvent is silicone oil, sunflower oil, lemon oil, an essential oil, paraffin oil, liquid paraffin, a fluorinated oil, carnation oil, an ionic liquid, mineral oil, an edible oils, or any combination thereof. In one aspect, the hydrophobic solvent is an alkane such as, for example, a branched or linear C6 to C20 alkane. In another aspect, the alkane is n-hexadecane.

In one aspect, the compositions described herein are produced by contacting the nitric oxide-releasing material with the nanoemulsion. Not wishing to be bound by theory, the nanoemulsion infuses into the polysiloxane network of the nitric oxide-releasing material.

In one aspect, the nitric oxide-releasing material is immersed in a composition comprising the nanoemulsion to produce the nitric oxide-releasing composition. The nitric-oxide releasing composition can subsequently be applied to an article such as a medical device (e.g., catheters, extracorporeal life support, tubing, cannulas).

In another aspect, a medical device can be coated with the nitric oxide-releasing material first to produce a first coated article. The first coated article can subsequently be contacted with the nanoemulsion to produce the nitric oxide-releasing article.

A common challenge that indwelling biomaterials face is biofouling due to bacterial or blood adhesion to the surface. The fouling material interacts with the biomaterial surface and accumulates until the material is no longer usable. One of the key components of an effective slippery coating on biomaterials is its longevity and resilience after interacting with foreign materials, such as bacteria or blood. One metric to assess a slippery coating is to measure a surface's sliding angle, the angle that a liquid droplet requires to slide down the surface. A surface is considered slippery if its sliding angle is equal to or less than 20 degrees,¹⁷ indicating low surface interactions. The compositions described herein have a sliding angle of less than 20 degrees, or a sliding angle of 10 degrees, 12 degrees, 14 degrees, 16 degrees, 18 degrees, or less than 20 degrees.

By varying the relative amount of the nitric oxide-releasing material and nanoemulsion, the rate of release of the nitric oxide releasing material from the composition can be modified. In certain applications, it is desirable to have sustained release of nitric oxide from the composition under physiological conditions. In one aspect, nitric oxide is released from the composition for at least 30 days, at least 45 days, at least 60 days, at least 75 days, or at least 90 days at 37° C. In another aspect, the amount of nitric oxide released from the composition is at least 0.5×10⁻¹⁰ mol cm⁻² min⁻¹ over a period of 5 days, 10 days, 15 days, 20 days, 25 days, or 30 days. In another aspect, the amount of nitric oxide released from the composition is from about 0.5×10⁻¹⁰ mol cm⁻² min⁻¹ to about 2.0×10¹⁰ mol cm⁻² min⁻¹ over a period of 5 days, 10 days, 15 days, 20 days, 25 days, or 30 days.

The use of biomedical devices is inevitable in hospital-based care. Unfortunately, it is also one of the leading causes of nosocomial infections. There are multiple factors that contribute to the risk of infection, including the method and duration of catheterization, quality of catheter care, and host susceptibility. The colonization of bacteria can cause infection through a number of pathways, the two most common being colonization at the insertion site by microorganisms that move through the transcutaneous part of the dermal tunnel surrounding the catheter and colonization in the intraluminal surface. Plasma proteins binding on the surface of intravascular catheters worsen this problem as they can promote bacterial adhesion. Fibrinogen is a positive acute phase protein related to blood infection, inflammatory disease, and tissue damage, ultimately causing patient mortality via trauma coagulopathy.

The compositions described herein are useful in applications where it is desirable to reduce or prevent biofouling (e.g., bacterial adhesion, platelet formation, etc.) of implantable medical devices. Implantable medical devices are a leading cause of infection such as nosocomial infections. Implantable devices coated with or constructed of the compositions described herein can reduce or prevent biofouling in a subject when the device is introduced into the subject.

In one aspect, the compositions described herein can reduce or prevent bacterial growth on a surface of an implantable device. In another aspect, the compositions described herein can reduce or prevent biofilm formation on a surface of an implantable device. Bacterial infection is one of the prevalent causes for implanted medical device failure in terms of morbidity and mortality. Infections caused by skin microflora create complications such as implanted device failure due to occlusion, adverse immune responses, and sepsis. The compositions described herein can help prevent infections altogether and/or combat existing infections.

Thrombosis is one of the major challenges that indwelling medical devices as an inherent bodily defense mechanism. Thrombosis is induced when a signaling cascade is triggered, platelets begin to aggregate to form a platelet plug, and the irreversible adhesion of proteins occurs. In indwelling medical devices such as catheters, thrombus formation can be especially detrimental to proper blood circulation and oxygenation; occlusion of the catheter lumen due to thrombus prevents adequate blood flow and drug administration, leaving the patient more susceptible to potential embolisms (e.g. pulmonary embolism). Traditional anticoagulant methods use systemic administration, which has adverse effects of excess bleeding, bruising, or thrombocytopenia (in the case of heparin).

In one aspect, the compositions described herein can reduce or prevent fibrinogen formation on a surface of an implantable device. Fibrinogen, a key coagulation protein, rapidly adsorbs to foreign surfaces and activates platelets. Fibrinogen contains multiple binding sites for platelet integrin α_{IIb}β₃ (GPIIbIIIa). These fibrinogen-α_{IIb}β₃ interactions play a significant role in platelet adhesion, activation, and aggregation that ultimately leads to a clot formation. To prevent thrombosis, a surface that can resist both fibrinogen binding and platelet activation is highly desirable; however, no surface reported to date is able to accomplish this.

Hemocompatibility of blood-contacting biomaterials is highly dependent on the suppression of both the contact coagulation pathway (i.e., fibrin formation) and the activation of circulating platelets. The nonthrombogenic surfaces that are currently available are designed to inhibit fibrin formation, but these surfaces do not prevent the parallel hemostatic pathway of platelet adhesion/activation and therefore are minimally effective. Thrombus and biofilm formation are highly related, where fouling of these devices is the most common clinical complication, either through protein adsorption leading to thrombus formation or bacterial adhesion resulting in infection. The compositions described herein can prevent platelet adhesion on a surface of an article.

In one aspect, the implantable device is a urinary catheter, artificial heart valve, a vascular catheter, a graft, extracorporeal life support, tubing, cannula, or a stent. In other aspects, the device is intended to contact human blood or tissue. In one aspect, the device is a hemodialysis device or a component thereof.

The compositions described herein can be incorporated into devices in a number of different ways. In one aspect, the devices can be coated with a composition as described herein. In one aspect, the coating composition can include an admixture of the nitric oxide-releasing material and silicone oil. In another aspect, a coating of the nitric oxide-releasing material can be applied to a surface of the device to produce a first coating followed by applying a coating of silicone oil on the first coating. The coating of the device can be performed using techniques known in the art such as, for example, spraying or dipping the device with the nitric oxide releasing material and silicone oil. The coating thickness can vary as well depending upon the device and application selected. In one aspect, the nitric oxide releasing-material coating has a thickness of from about 0.1 mm to about 5 mm, or about 0.1 mm, 0.5 mm, 1.0 mm, 1.5 mm, 2.0 mm, 2.5 mm, 3.0 mm, 3.5 mm, 4.0 mm, 4.5 mm, or 5.0 mm, where any value can be a lower and upper endpoint of range (e.g., 0.5 mm to 3.0 mm).

In other aspects, the compositions described herein can be used to fabricate a device. For example, when the device is composed of rubber or includes a rubber component, the rubber can be prepared such that the composition described herein is dispersed throughout the rubber to produce a nitric oxide-releasing rubber. Once the nitric oxide releasing rubber has been produced, it can be used to produce devices (e.g., medical implantable devices).

In another aspect, the compositions described herein can include one or more bioactive agents. In one aspect, the dispersed water droplets from the nanoemulsion can be used to load water-soluble agents for drug diffusion, allowing for a wider range of efficacy. For example, a bioactive agent in water can be mixed with the surfactant and a hydrophobic solvent to produce nanoemulsions loaded with the agent. In one aspect, the bioactive agent can be an anti-bacterial agent or an anti-inflammatory agent.

### ASPECTS

Aspect 1. A composition comprising
(a) a nanonemulsion comprising a surfactant and a hydrophobic solvent; and
(b) a nitric oxide releasing material comprising a (i) a polysiloxane network and (ii) a plurality of nitric oxide-donating moieties covalently bonded to the polysiloxane network.

Aspect 2. The composition of Aspect 1, wherein the surfactant comprises a nonionic surfactant.

Aspect 3. The composition of Aspect 1, wherein the surfactant comprises two different nonionic surfactants.

Aspect 4. The composition of Aspect 2 or 3, wherein the nonionic surfactant comprises polyoxyethylene (20) sorbitan monolaurate, sorbitan monolaurate, polyoxyethylene 2-lauryl ether, polyoxyethylene 4-lauryl ether, polyglycerol polyricinoleate, or any combination thereof.

Aspect 5. The composition of Aspect 1, wherein the surfactant comprises polyoxyethylene (20) sorbitan monooleate and sorbitan monooleate.

Aspect 6. The composition of Aspect 5, wherein the weight ratio of sorbitan monooleate to polyoxyethylene (20) sorbitan monooleate is from 2:1 to 4:1.

Aspect 7. The composition of any one of Aspects 1-6, wherein the hydrophobic solvent comprises an alkane, silicone oil, sunflower oil, lemon oil, an essential oil, paraffin oil, liquid paraffin, a fluorinated oil, carnation oil, an ionic liquid, mineral oil, an edible oils, or any combination thereof.

Aspect 8. The composition of Aspect 7, wherein the alkane is a C6 to C20 alkane.

Aspect 9. The composition of any one of Aspects 1-6, wherein the hydrophobic solvent comprises n-hexadecane.

Aspect 10. The composition of any one of Aspects 1-9, wherein the weight ratio of the hydrophobic solvent to surfactant is from 1:1 to 3:1.

Aspect 11. The composition of Aspect 1, wherein the surfactant comprises polyoxyethylene (20) sorbitan monooleate and sorbitan monooleate and the hydrophobic solvent comprises n-hexadecane.

Aspect 12. The composition of Aspect 11, wherein the weight ratio of sorbitan monooleate to polyoxyethylene (20) sorbitan monooleate is from 2:1 to 4:1, and the weight ratio of n-hexadecane to the sum of polyoxyethylene (20) sorbitan monooleate and sorbitan monooleate is from 1:1 to 3:1.

Aspect 13. The composition of any one of Aspects 1-12, wherein the nitric oxide-donating moiety comprises an S-nitrosothiol.

Aspect 14. The composition of any one of Aspects 1-12, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

Aspect 15. The composition of any one of Aspects 1-14, wherein the polysiloxane network comprises a polysiloxane crosslinked with an amine-functionalized crosslinker.

Aspect 16. The composition of Aspect 15, wherein the polysiloxane comprises a polydimethylsiloxane, a polydiethylsiloxane, a polydipropylsiloxane, or a polydiphenylsiloxane.

Aspect 17. The composition of Aspect 15, wherein the polysiloxane has a kinematic viscosity of about 2,500 cSt to about 4,000 cSt.

Aspect 18. The composition of any one of Aspects 1-17, wherein the nitric oxide-releasing material is produced the method comprising:
crosslinking a polysiloxane with an amine-functionalized crosslinker to produce a polysiloxane network
covalently attaching a thiolactone to the polysiloxane network to produce a thiol-functionalized polysiloxane network; and
nitrosating a thiol group in the thiol-functionalized polysiloxane network to produce the nitric oxide-releasing material.

Aspect 19. The composition of Aspect 18, wherein the polysiloxane comprises a hydroxy-terminated polysiloxane.

Aspect 20. The composition of Aspect 18 or 19, wherein the amine-functionalized crosslinker has the structure

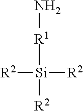

where R¹ is selected from a substituted or unsubstituted C₁-C₂₀ alkyl, a substituted or unsubstituted C₁-C₂₀ heteroalkyl, a substituted or unsubstituted C₂-C₂₀ alkenyl, a substituted or unsubstituted C₂-C₂₀ heteroalkenyl, a substituted or unsubstituted C₁-C₂₀ alkoxy, or a substituted or unsubstituted C₁-C₂₀ heteroalkoxy;
where each occurrence of R² is hydroxy or alkoxy.

Aspect 21. The composition of Aspect 20, wherein each occurrence of R² is a hydroxy, methoxy or ethoxy.

Aspect 22. The composition of Aspect 20, wherein R¹ is a substituted or unsubstituted C₁-C₁₂ alkyl or a substituted or unsubstituted C₁-C₁₂ aminoalkyl.

Aspect 23. The composition of Aspect 20, wherein R¹ is methylene, ethylene, propylene, or butylene.

Aspect 24. The composition of any one of Aspects 18-23, wherein the thiolactone has the structure

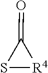

where R⁴ is a substituted or unsubstituted C₁-C₁₂ alkyl.

Aspect 25. The composition of any one of Aspects 18-23, wherein the thiolactone has the structure

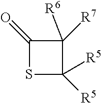

where each occurrence of R⁵ is independently hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, or a substituted or unsubstituted C₁-C₆ heteroalkoxy group;
R⁶ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, or a substituted or unsubstituted C₁-C₆ heteroalkoxy group; and
R⁷ is hydrogen, a hydroxyl group, a substituted or unsubstituted C₁-C₆ alkyl group, substituted or unsubstituted C₁-C₆ heteroalkyl group, a substituted or unsubstituted C₂-C₆ alkenyl group, a substituted or unsubstituted C₂-C₆ heteroalkenyl group, a substituted or unsubstituted C₁-C₆ alkoxy group, a substituted or unsubstituted C₁-C₆ heteroalkoxy group, or an amide group of the formula —NHC(O)R⁸, wherein R⁸ is a substituted or unsubstituted C₁-C₆ alkyl group, substituted or unsubstituted C₁-C₆ heteroalkyl group.

Aspect 26. The composition of any one of Aspects 18-23, wherein the thiolactone is selected from the group consisting of N-acetylcysteine thiolactone, N-acetyl-homocysteine thiolactone, homocysteine thiolactone, and butyryl-homocysteine thiolactone.

Aspect 27. The composition of Aspect 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, or methyl S-nitrosothioglycolate.

Aspect 28. The composition of any one of Aspects 1-27, wherein the nitric oxide-donating moieties are present in an amount from about 0.15 micromoles per milligram of the polymer matrix to about 0.80 micromoles per milligram of the polysiloxane network.

Aspect 29. The composition of any one of Aspects 1-28, wherein the weight ratio of the nanoemulsion to the nitric oxide releasing material is from 1:1 to 2:1.

Aspect 30. The composition of any one of Aspects 1-28, wherein the composition is produced by contacting the nitric oxide releasing material with the nanoemulsion.

Aspect 31. The composition of Aspect 30, wherein the nitric oxide releasing material is immersed in a composition comprising the nanoemulsion and water.

Aspect 32. The composition of any one of Aspects 1-31, wherein nitric oxide is released from the composition for at least 7 days at 37° C.

Aspect 33. The composition of any one of Aspects 1-31, wherein the composition has a sliding angle of less than 20 degrees.

Aspect 34. The composition of any one of Aspects 1-31, wherein the composition comprises an admixture of the nitric oxide releasing material and the nanoemulsion.

Aspect 35. The composition of any one of Aspects 1-31, wherein the nitric oxide releasing material is infused with the nanoemulsion.

Aspect 36. The composition of any one of Aspects 1-35, wherein the composition further comprises a bioactive agent.

Aspect 37. An article comprising at least one surface, wherein the at least one surface is coated with the composition in any one of Aspects 1-36.

Aspect 38. An article comprising one or more components fabricated with the composition in any one of Aspects 1-36.

Aspect 39. The article of Aspect 37 or 38, wherein the article comprises a medical device.

Aspect 40. The article of Aspect 39, wherein the device is an implantable device.

Aspect 41. The article of Aspect 39, wherein the device is selected from the group consisting of: a vascular catheter, a urinary catheter, other catheters, a coronary stent, a wound dressing, and a vascular graft.

Aspect 42. A method of making an article, the method comprising
(a) applying to at least one surface of the article a nitric oxide releasing material comprising a (i) a polysiloxane network and (ii) a plurality of nitric oxide-donating moieties covalently bonded to the polysiloxane network to produce a first coated article; and
(b) applying a nanonemulsion comprising a surfactant and a hydrophobic solvent to the first coated article.

Aspect 43. The method of Aspect 42, wherein the nitric oxide releasing material has a thickness of from about 0.1 mm to about 5 mm.

Aspect 44. The method of Aspect 42, wherein the first coated article is dipped into the nanoemulsion from about 1 hour to about 12 hours.

Aspect 45. A method of making an article, the method comprising applying to at least one surface of the article the composition in any one of Aspects 1-36.

Aspect 46. A method of preventing bacterial growth on a surface of an article, the method comprising applying the composition in any one of Aspects 1-36 to the surface.

Aspect 47. A method of preventing biofilm formation on a surface of an article, the method comprising applying the composition in any one of Aspects 1-36 to the surface.

Aspect 48. A method of preventing fibrinogen formation on a surface of an article, the method comprising applying the composition in any one of Aspects 1-36 to the surface.

### Examples

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Methods and Materials

### Materials

N-acetyl-D-penicillamine (NAP, ≥99.0%), anhydrous pyridine (99.8%), chloroform (99.5%), hexanes (mixture of isomers, 98.5%), anhydrous magnesium sulfate (99.5%), poly(dimethylsiloxane), hydroxy-terminated, 2550-2570 cSt, (3-aminopropyl) trimethoxysilane (APTMS, 96.0%), dibutyltin dilaurate (DBTDL, 95%), toluene (99.5%), dimethyl sulfoxide (DMSO, 99.9%), tert-butyl nitrite (TBN, 90%), n-hexadecane (99%), thiazolyl blue tetrazolium bromide 98%, sodium citrate dihydrate, fibrinogen from human plasma (>90%), and Luria Bertani (LB) broth media and agar were purchased from Sigma Aldrich (St. Louis, MO) for sample fabrication and biological studies, namely bacterial adhesion assays and cytocompatibility evaluation. Acetic anhydride (99%) was purchased from Alfa Aesar (Haverhill, MA) for sample fabrication. Drabkin's Reagent was purchased from Ricca Chemical Company for the hemolysis assay (Arlington, TX). Human Fibrinogen Purified, FITC Labeled (>95%) was purchased from Innovative Research (Novi, MI) and used for the fibrinogen adsorption assay. All aqueous solutions were prepared using deionized water. Phosphate buffer saline (PBS) 10 mM, containing sodium chloride (139 mM), potassium chloride (2.68 mM), sodium phosphate monobasic (1.8 mM), sodium phosphate dibasic (8.2 mM), ethylenediaminetetraacetic acid (EDTA, 100 μM) and adjusted to pH 7.4, was used for all material characterization and nitric oxide analyzer (NOA) studies. Calcium and magnesium-free PBS (1×) was purchased from Corning Incorporated (Manassas, VA). Dulbecco's Modified Eagle Medium (DMEM), fetal bovine serum (FBS), and penicillin-streptomycin (P/S) were purchased from VWR (Radnor, PA). The lactic dehydrogenase (LDH) kit was purchased from Roche Life Sciences (Indianapolis, IN). Bacterial strains Staphylococcus aureus (S. aureus, ATCC 6538) and Escherichia coli (E. coli, ATCC 25922) for the bacterial adhesion assay and 3T3 mouse fibroblast cells (ATCC 1658) for cell compatibility were obtained from American Type Culture Collection (ATCC, Manassas, VA). Fresh porcine whole blood was acquired from the University of Georgia Swine Unit (Athens, GA) and used for the platelet adhesion assay and hemolysis assay.

### SNAP-PDMS Fabrication

### N-Acetyl Penicillamine-Thiolactone (NAPTH) Synthesis

N-acetyl penicillamine-thiolactone (NAPTH) was prepared following a previously reported protocol with some minor deviation.³² First, 5 g of N-acetyl-D-penicillamine (NAP) was dissolved in 10 mL of pyridine in a round-bottom flask and allowed to stir for 30 min in an ice bath. While the NAP was being dissolved, 10 mL of pyridine was mixed with 10 mL of acetic anhydride in a separate vial and chilled in an ice bath. The pyridine and acetic anhydride mixture (20 mL) was added to the round-bottom flask, purged with argon with a supplemented balloon, protected from light, and stirred for 12-15 h. The reacted solution turned a rose-gold color and was rotary evaporated using an IKA RV 10 auto rotary evaporator (IKA, Wilmington, NC) at 40° C. to remove the solvent. Once the pot was completely dry, the resulting NAPTH was resuspended in ~20 mL of chloroform and washed 3 times with equal volume quantities of 1 M hydrochloric acids (HCl). The organic phase was extracted and filtered over anhydrous magnesium sulfate. The filtered solution was added to 300 mL of chilled hexanes and chilled at −20° C. overnight. The solution was then vacuum filtered and washed with hexanes (isomeric mixture). The resulting precipitate was white with a pale yellow tinge and dried overnight. After drying, the NAPTH crystals were stored at −20° C. and protected from light.

### SNAP-PDMS Film Fabrication

Covalently attached SNAP-PDMS was synthesized using a modified protocol.²⁵ Briefly, 1600 mg of hydroxy-terminated PDMS was dissolved in 8 mL of H₂O-saturated toluene (H₂O-toluene) in a 20 mL scintillation vial. In 2 separate vials, 300 mg of (3-aminopropyl) trimethoxysilane (APTMS) was dissolved in 1 mL of H₂O-toluene, and a dibutyltin dilaurate (DBTDL) solution (2.4 mg mL⁻¹) was prepared. Equal volumes (1 mL) of the APTMS solution and the DBTDL solution were added to the PDMS solution. The solution was stirred for 24 h at room temperature. Then, 300 mg of NAPTH was added to the crosslinked PDMS solution and allowed to stir for 48 h. Tert-butyl nitrite (TBN) was chelated of copper contaminants by washing with a 25 mM solution of cyclam-B. The polymer solution (9 mL) was transferred to a new 20 mL scintillation vial, and 1.5 mL of cleaned TBN was added to the solution to S-nitrosate the NAP-PDMS. 4-dodecylbenzenesulfonic acid (DBSA, 10 μL) was added to the nitrosated solution. The nitrosated solution was then protected from light and stirred for an additional 5 min at room temperature. Finally, the polymer solution was cast into a 7×7 cm Teflon mold, protected from light, dried under ambient conditions for 24 h, and vacuum dried for another 24 h. The thickness of the SNAP-PDMS samples were measured with a micrometer. The resulting polymer film was stored at −20° C. for further experiments.

### Sylgard 184 PDMS Film Fabrication

Sylgard 184 PDMS films, herein referred to as PDMS, were also fabricated as the control. Briefly, the Sylgard 184 Elastomer base was mixed with the curing agent in a 10:1 ratio. Anhydrous tetrahydrofuran (THF) was added to the Elastomer-curing agent mixture to decrease the solution viscosity. The PDMS solution was then cast into a 7×7 cm Teflon mold, dried under ambient conditions for 24 h, and vacuum dried for another 24 h to remove residual solvent. The thickness of the PDMS samples were measured with a micrometer. The PDMS film was stored at room temperature.

### Nanoemulsion (NE) Synthesis, Characterization, and Polymer-NE Infusion

### Synthesis

The NE was synthesized by modifying a protocol by Agarwal et al.²⁸ Briefly, polyoxyethylene (20) sorbitan monooleate (Tween 80; 7.5 parts by weight) and sorbitan monooleate (Span 80; 22.5 parts by weight) was dissolved in n-hexadecane (70 parts by weight) and vortexed to thoroughly mix. The surfactant mixture in n-hexadecane was vacuum-filtered through a 0.1 μm PTFE filter. Deionized water (5% v/v) was added to a vial, and the surfactant mixture dissolved in n-hexadecane was added to the vial at a rate of 200 μL per 20 s with continuous stirring at 700 rpm. The resulting NE was left to stir 30 min at room temperature. Then, the NE was sonicated using an ultrasonic probe (Split Type Ultrasonic Processor, Vevor, Shanghai, China, 50%, 19.5 kHz) for 2.5 min. The NE was left to stir at 700 rpm for 1 h. After sonication and additional stirring, the NE remained stable.

### Dynamic Light Scattering (DLS)

The hydrodynamic diameter of the NE droplets was evaluated using dynamic light scattering (DLS), which is based on the Brownian motion of dispersed particles. As the Brownian motion of the particles increases, so does the amount of light scattering that occurs, changing the intensity of the measured light.³³ The formed NE was diluted with n-hexadecane in a 1:200 ratio; the diluted NE was processed with the Zetasizer Nano ZS (Malvern Panalytical, Malvern, Worcestershire, United Kingdom) over 3 measurements to confirm the formation of a nanoemulsion (with hydrodynamic diameters <100 nm).

### Polymer-NE Infusion

To determine an adequate amount of time for polymer films to infuse with the NE, the swelling ratios and rates were calculated. Essentially, each sample type was first weighed and immersed in the NE. The weight of each sample type was recorded over a period of 24 h, with measurements every 2 hours; the swelling ratios and swelling rates were calculated according to Equations 1 and 2, respectively, where mx is the mass of the sample at a certain time point, mᵢₙᵢₜᵢₐₗ is the mass of the sample prior to immersion in the nanoemulsion, ΔSR is the change in swelling ratio, and ΔTime is the change in time. The swelling ratio and rate stabilized after 4 h; therefore, to infuse the NE into polymers, PDMS and SNAP-PDMS were immersed in the NE for 4 h. The resulting NE-swelled polymer films, referred to as PDMS-NE and SNAP-PDMS-NE, were then used for further studies along with their controls, PDMS and SNAP-PDMS, respectively. The final weights of all sample types were recorded.

$$Swelling Ratio ( SR ) = \frac{{m}_{x}}{{m}_{initial}} \qquad ( 1 )$$

$$Swelling Rate = \frac{Δ SR}{ΔTime} \qquad ( 2 )$$

### Fourier Transform Infrared Spectroscopy (FTIR)

Fourier transform infrared spectroscopy (FTIR) was conducted using a Spectrum 3 spectrometer (Perkin-Elmer, Waltham, MA) with universal attenuated total reflectance (UATR) accessory to verify the conjugation of SNAP onto hydroxy-terminated PDMS and to confirm the presence of the nanoemulsion. UATR-FTIR was used to study the composition of the liquid lubricant n-hexadecane as well as surfactants Span 80 and Tween 80 for comparison to the polymer composites. Samples were studied across a spectral range of 4000 to 650 cm⁻¹ with a resolution of 4 cm-1. A total of 64 scans were collected for each sample. Spectra were processed using Perkin-Elmer Spectrum 10 software.

### NE Coating Stability

### Static Water Contact Angle

Evaluating the static water contact angle of the samples provides insight to the surface wettability and was measured with a Contact Angle Goniometer (Ossila Ltd., Sheffield, England). Briefly, all control and test groups (PDMS, SNAP-PDMS, PDMS-NE, and SNAP-PDMS-NE; n=3) were placed on the goniometer stage, and the camera was focused. Two 2 μL droplet of DI water was placed on the surface of the sample, allowed to stabilize, and the static water contact angle was measured in the Ossila Contact Angle software. This was repeated for all technical replicates.

### Sliding Angle Measurement

In addition to the static water contact angle, surface wettability can be further assessed by measuring the sliding angle. All control and test groups (PDMS, SNAP-PDMS, PDMS-NE, and SNAP-PDMS-NE; n=3) were measured. Each film was placed on a digital protractor. A droplet of toluidine blue-dyed DI water (1 mg mL⁻¹) was placed on the film, and the digital protractor was incrementally changed 1° at a time until the water droplet began to slide on the film. The angle at the moment when the water droplet began to slide was recorded as the sliding angle. For PDMS-NE and SNAP-PDMS-NE, the polymer film was taken out of the NE, and the excess NE was gently dried with a cellulose wipe prior to measuring the sliding angle. Then the sliding angle was measured as described.

### NE Coating Stability Over 7 Days

To determine the stability of the NE coating, the sliding angle of the NE-swelled polymers (PDMS-NE, SNAP-PDMS-NE; n=3) was measured over a 7-d period. After measuring the initial sliding angle (Day 0), the films were submerged in 10 mM phosphate-buffered saline (PBS) with 100 μM EDTA and incubated at 37° C. and 150 rpm for 24 h. The films were taken out of the PBS, gently dried with a cellulose wipe, and the sliding angle was measured as described above in Section 2.4.2. The films were placed back in fresh PBS and incubated on a VWR Mini Incubated Shaker (VWR, Radnor, PA). This process was repeated every other day until the end of the 7-d period.

### Nitric Oxide Donor Loading

The loading of the NO donor SNAP was measured by catalyzing all of the NO out of the covalently bound SNAP-PDMS (4.78 mg) using a modified protocol.³⁴ Using a hot plate at 130° C. (Corning, NY) and light irradiation from a commercial flashlight, the NO release from the SNAP-PDMS was measured in a dry state (i.e., without immersion in 10 mM PBS w/100 μM EDTA) via chemiluminescence using a Zysense Nitric Oxide Analyzer (NOA) 280i (Frederick, CO). The moles of NO released was calculated and correlated to the moles of the NO donor SNAP within the polymer (i.e., 1 mol SNAP releases 1 mol NO).

### Nitric Oxide Detection

The NO released from the SNAP-PDMS and SNAP-PDMS-NE films (n=4) were measured via chemiluminescence using a gold-standard Zysense Nitric Oxide Analyzer (NOA) 280i (Frederick, CO). The NO-releasing films were first submerged in 10 mM PBS with 100 μM EDTA at 37° C. in an amber sample chamber. The dark, warm, and saline conditions (similar pH and salt concentrations as human bodily fluids) were ideal for this experiment because they mimic physiological conditions that an implanted medical device may experience. A nitrogen bubbler and sweep gas were placed in the solution with the NO-releasing film at a flow rate of 200 mL min- to take any NO released in solution to the NOA. The samples were measured until a steady NO release state was reached. Each NO-releasing sample was tested every 2 d over a 14-d period. The samples were then incubated in PBS at 37° C. protected from light between time points.

### Bacterial Adhesion Assay

The antibacterial efficacy of the SNAP-PDMS-NE samples was evaluated against the bacterial strains S. aureus ATCC 6538 (Gram positive) and E. coli ATCC 25922 (Gram negative) in a 24 h antibacterial adhesion assay following previously reported protocols.³⁵ Briefly, an individual colony of bacteria was isolated from a cultured agar plate and inoculated in LB broth until mid-log phase growth. The optical density of the culture was measured at a wavelength of 600 nm using a UV-vis spectrophotometer (Thermo Scientific Genesys 10S UV-vis) to verify the growth. The cells were then washed with PBS, and the final concentration was adjusted to 0.1 optical density prior to polymer exposure. The PDMS, PDMS-NE, SNAP-PDMS, and SNAP-PDMS-NE samples were then incubated in the bacterial suspension for 24 h (37° C., 150 rpm). After 24 h, the samples were rinsed to remove loosely adhered bacteria and resuspended in sterile PBS. The adhered bacteria were then mechanically detached from the samples using an Omni-TH homogenizer (Omni, Kennesaw, GA) for 60 s, followed by another 60 s of vortex to remove adhered bacteria from the surfaces. Bacterial suspensions were plated on LB agar plates using a bacteria spiral plater (Eddy Jet 2W, IUL instruments) and incubated at 37° C. overnight. The viable colony forming units (CFUs) were counted, and bacterial adhesion was calculated and analyzed as CFU cm⁻² according to Equation 3, where CFU_{control} is the number of CFUs adhered to control PDMS and CFUₛₐₘₚₗₑ is the number of CFUs on the test groups (i.e. PDMS-NE, SNAP-PDMS, SNAP-PDMS-NE). Final data are reported as the mean±standard deviation (n 5 samples).

$$\% Bacterial Reducation = \frac{{CFU}_{control} - {CFU}_{sample}}{{CFU}_{control}} * 100 \qquad ( 3 )$$

### In Vitro Hemocompatibility

### Platelet Adhesion Assay

To evaluate the anti-platelet capabilities of the NO-releasing slippery surface, platelet adhesion on all controls and test groups were assessed via LDH assay. Briefly, fresh porcine whole blood was collected and citrated in a 1:9 citrate to whole blood ratio to prevent the blood from coagulating. The whole blood was then centrifuged to separate the red blood cells, the buffy layer, and the plasma. The platelet rich plasma (PRP) was collected, diluted with platelet poor plasma (PPP), and reverse citrated with 200 mM CaCl₂ (to achieve a final concentration of 2.5 mM CaCl₂) to a working concentration of 2×10⁸ platelets mL-1. The control and test group samples were incubated in the dilute PRP for 90 min at 37° C. with gentle rocking. The samples were manually inverted every 30 min during the 90 min incubation period. After the incubation period, the samples were removed from the dilute PRP and washed with calcium and magnesium free phosphate buffered saline (CMF-PBS) to remove any non-adhered platelets. The samples were then incubated in Triton X-100 for 30 min to lyse the platelets. The lysing solution was then plated and combined with Roche solution. The absorbance of the plate was then read at 492 nm and 620 nm with a BioTek (Winooski, VT) plate reader and translated into concentration of platelets adhered on to each surface. Final data are reported as the mean±standard deviation (n=8).

### Hemolytic Evaluation

The hemolytic activity of SNAP-PDMS-NE samples was evaluated against control samples using a direct contact method following ISO 10993-4 guidelines.³⁷ Porcine whole blood (WB) was collected and treated with sodium citrate dihydrate to prevent premature clotting. The hemoglobin (Hgb) concentration in the WB was measured and diluted with CMF-PBS (1×) to achieve a final working concentration of 10 mg mL⁻¹. Samples were incubated in the diluted whole blood (DWB) at an extraction ratio of 3 cm² mL⁻¹ for 3 hours at 37° C., with manual inversions every 30 minutes.

After incubation, samples were removed, and the DWB was centrifuged to separate intact blood cells from lysed red blood cells in the supernatant. The supernatant was then mixed with Drabkin's reagent in a 1:1 ratio and incubated for 15 minutes at room temperature. Absorbance was measured at 540 nm using a BioTek Cytation 5 Imaging Reader (Winooski, VT). A standard curve was prepared by dissolving porcine Hgb in CMF-PBS (1×), performing serial dilutions, and measuring absorbance at 540 nm. The hemoglobin concentration from the samples was calculated using the standard curve, and % hemolysis was determined using Equation 5, where [Hgb]ₛₐₘₚₗₑ is the concentration of the Hgb in the supernatant after sample incubation in DWB, [Hgb]_{baseline} is the concentration of Hgb in the DWB supernatant without lysing agents, and [Hgb]ₜₒₜₐₗ is the total amount of Hgb in the DWB with lysing agents. The hemolytic index (HI) of the samples was calculated according to Equation 6. The final results are reported as a mean±standard deviation (n=6, p<0.05).

$$\% Hemolysis = \frac{{[ Hgb ]}_{sample} - {[ Hgb ]}_{baseline}}{{[ Hgb ]}_{total}} * 100 \qquad ( 5 )$$

$$Hemolytic Index ( HI ) = \% {Hemolysis}_{sample} - \% {Hemolysis}_{NegControl} \qquad ( 6 )$$

### Protein Adsorption Evaluation

Fibrinogen (Fg) adsorption onto samples was evaluated through incubation with FITC-labeled fibrinogen (FITC-Fg). Briefly, the SNAP-PDMS-NE and controls (n=8) were incubated with CMF-PBS at 37° C. for 1 h in a 96-well black polypropylene plate to reach surface saturation. After 1 h, a background scan of the plate was collected on a Biotek Plate Reader (Winooski, VT) with excitation at 490/20 and emission at 525/20. Then, a working concentration (4 mg mL-1) of Fg was prepared consisting of 1:10 FITC-Fg to unlabeled Fg.

A standard curve was prepared with the diluted Fg in a 1:1 serial dilution. Each dilution was plated in triplicate. Then, 100 μL of the diluted Fg solution was added to each sample well without removing the initial CMF-PBS and incubated at 37° C. for 90 min. After the incubation, each sample well was washed several times (8-25 times) with 100 μL of CMF-PBS. The fluorescence intensity was then read on the Biotek Cytation 5 Imaging Reader (Ex: 490/20, Em: 525/20). The amount of Fg adsorbed onto the surface was calculated and normalized to the surface area. The final results are reported as a mean±standard deviation (n=8, p<0.05).

### In Vitro Cell Cytocompatibility

### 3T3 Mouse Fibroblast Cell Culturing

Cell cytocompatibility was assessed for the SNAP-PDMS-NE films using NIH 3T3 mouse fibroblast cells per the ISO 10993-5 standard.³⁶ The 3-(4,5-dimethylthiazol-2-yl)-2,5-diphenyl-2H-tetrazolium bromide (MTT) assay protocol was employed using the leachate exposure method. The mouse fibroblast cells were cultured on T75 (75 cm²) flasks with complete Dulbecco's Modified Eagle Medium (DMEM with 10% fetal bovine serum and 1% penicillin streptomycin) at 37° C. and 5% CO₂. When the cells were mature and confluent, they were seeded on a sterile 96-well plate at a cell seeding density of 1×10⁴ live cells well⁻¹ to adhere.

### Leachate Preparation

All control and test groups (n=4) were first sterilized with 70% ethanol and UV-sterilized for 30 min. After the sterilization, the leachates were prepared by immersing the films in complete DMEM as the extraction vehicle for 24 h at 37° C. and 5% CO₂. After 24 h, the films were removed, and the leachates were used for cytocompatibility studies.

### Cytocompatibility of SNAP-PDMS-NE

After 24 h cell exposure to the prepared leachates, the leachates were removed from each well, and the diluted MTT reagent was added to each well of the plate and incubated at 37° C. and 5% CO₂ for 2.5 h. When the bottom of the wells began to exhibit purple and blue formazan crystals, the MTT solution was removed. The formazan crystals were re-dissolved in dimethyl sulfoxide (DMSO), and the absorbance was read with a BioTek Cytation 5 Imaging Reader (Winooski, VT) at 570 nm and 690 nm. The absorbance was translated into a relative percent viability of the fibroblast cells (Equation 4), where ABS(sample) is the absorbance of the cells exposed to test sample leachates (i.e. PDMS-NE, SNAP-PDMS, SNAP-PDMS-NE), ABS(control) is the absorbance of the cells exposed to control PDMS leachates, and ABS(blank) is the absorbance of the 96 well plate. Final data are reported as the mean±standard deviation (n=4 samples).

$$Relative Cell Viability ( \% ) = \frac{ABS ( sample ) - ABS ( blank )}{ABS ( control ) - ABS ( blank )} \qquad ( 4 )$$

### Statistical Analysis

All experiments conducted in this study are presented in n 3. All statistical analyses were performed using GraphPad Prism 9 (GraphPad Software, San Diego, CA). Data are presented as mean±standard deviation, and statistical significance was determined using a one-way ANOVA test. A p value<0.05 was used to determine statistical significance.

### Results and Discussion

### Material Fabrication

### SNAP-PDMS Fabrication

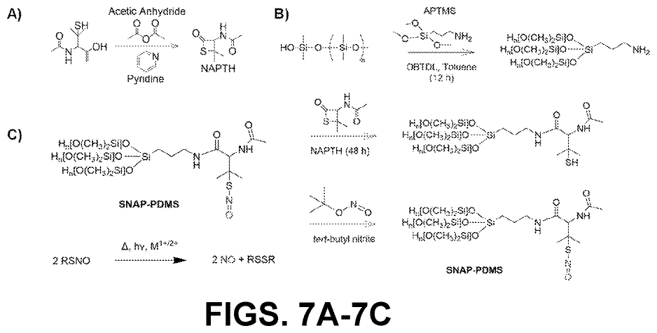

As a polymer, SNAP-PDMS is considered a very stable material in that the NO release profile has been proven to be prolonged for four months while maintaining antibacterial activity.²⁵ Literature has reported non-covalently dispersed SNAP in polymers achieved by directly blending SNAP in the polymer solution or a solvent swelling-impregnation method; however, the present SNAP-PDMS utilizes chemical conjugation of NAPTH (FIG. 7A) to covalently attach the NO donor precursor to the PDMS polymer matrix (FIG. 7B), providing more stability via chemical bonds compared to the blended or impregnated SNAP polymer equivalents.^{21,25,38} Where blended and impregnated SNAP materials capitalize on the SNAP that is crystallized between the polymer chains for steady NO release, these materials still require techniques that mitigate premature NO release and donor leaching.^{21,22,39} Covalent SNAP-PDMS uses hydroxy-terminated PDMS as the base polymer due to its silanol end group that condenses with the aminosilane crosslinker, wherein the NAPTH is then covalently attached by a thiolactone ring opening reaction.²⁵ The nitrosation of the conjugated NAP results in a stable silicone rubber-based NO-releasing material (SNAP-PDMS) that does not require such strategies to prevent NO donor leaching as it is negligible due to the covalent bonds.²⁵ A non-nitrosated cured version of hydroxy-terminated PDMS film was not feasible to directly compare to the nitrosated cured SNAP-PDMS film; therefore, Sylgard 184 PDMS was used as the next best PDMS control.

### Nanoemulsion (NE) Synthesis

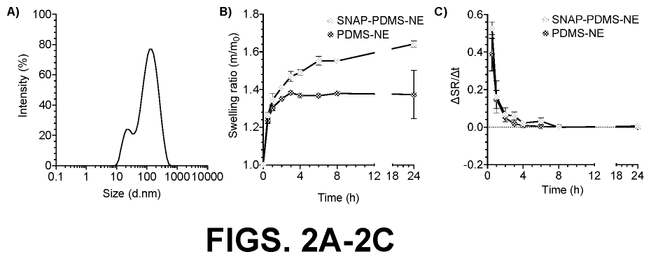

Nanoemulsions are emulsions of immiscible liquids with nano-sized droplets dispersed through the bulk phase, typically characterized by their translucent appearance, with dispersed droplets whose sizes are in the 20-200 nm range.⁴⁰ To create an NE, external forces, such as shear, are required to ensure thorough dispersion of the diffused phase and stability of the NE.²⁹ The necessary shear force was applied during the NE synthesis using magnetic stirring at 700 rpm to create an initial rough microemulsion in combination with ultrasonic agitation via an ultrasonic probe to cause cavitation of the micro-droplets, thus forming nano-sized water droplets encapsulated by surfactants. This combination produced a stable and evenly dispersed NE with a droplet size of 147.7 nm, verified through dynamic light scattering (DLS) analysis (FIG. 2A). After being left at room temperature for multiple days, the NE remained stable, and there were no signs of emulsion disruption or coalescence, namely turbidity or a phase separation. The non-polar co-surfactant system (Tween 80 and Span 80) contributed to the NE's stability as they act as a stabilizing micelle around the dispersed water droplets to prevent droplet collapse within the n-hexadecane.²⁸ The ratio of surfactants chosen has been shown to provide the long-term stability necessary to maintain a viable NE at room temperature;⁴¹ the stability in the solution phase demonstrates the stability when infused in a polymer.

### Polymer-Nanoemulsion Infusion

Polymers infused with silicone oil are typically silicone-based, allowing for the silicone backbone to interact with the silicone oil; however, as the NE used in this study was alkane-based (n-hexadecane), there was less chemical interaction between the oil and polymer chains. Despite the difference in chemical composition, the NE could still swell into the polymer matrix through physical liquid infusion and capillary action. Although there was an observed difference in swelling ratios between PDMS-NE and SNAP-PDMS-NE (FIG. 2B), which may be attributed to the difference in polymers used for each material, it was determined that 4 h was the optimal time for the NE to infuse into the Sylgard 184 PDMS and SNAP-PDMS polymer matrix without significantly changing the size of the sample (FIGS. 2B-C), which is less time than silicone oil infusion.⁴² This study's measured time period is not directly comparable to the swelling time that Agarwal et al. employed as they used PTFE porous membranes as their substrate and only infused the NE for 5 min.²⁸ This reduced swelling time measured in this study is most likely due to the lack of chemical interaction, as described previously. However, despite the shortened swelling time, the NE-swelled polymers, PDMS-NE and SNAP-PDMS-NE, maintained polymer integrity without sacrificing slipperiness. Although the swelling time is relatively short, and the NE reservoir within the polymer matrix is finite, the reservoir is sufficient to replenish any surface lubrication lost to agitation in a liquid or physical removal over the course of multiple days.

### Fourier Transform Infrared Spectroscopy (FTIR) Characterization

UATR-FTIR spectroscopy was used to study the chemical composition of PDMS, PDMS-NE, SNAP-PDMS, and SNAP-PDMS-NE. All materials showed characteristic peaks associated with polysiloxane including Si—O—Si stretching (1080-1020 cm⁻¹), Si—CH₃ stretching (1260 cm⁻¹), and C—H stretching (2960-2900 cm-1). Conjugation of SNAP onto the aminosilane crosslinker and its nitrosation was confirmed through emergence of new peaks at 1653 cm⁻¹ (C═O stretching) and 1548 cm⁻¹ (N—H stretching), consistent with prior findings.⁴³

Comparing PDMS-NE and SNAP-PDMS-NE to their parent materials, new peaks are observed consistent with the absorption bands of the n-hexadecane lubricant as well as the Span 80 and Tween 80 surfactants. Broad bands from 3300-3400 cm⁻¹ suggest O—H stretching which is consistent with Span 80 and Tween 80, while the presence of the n-hexadecane lubricant is supported by enhanced aliphatic character from 3000-2800 cm⁻¹ as well as weak peaks from 1300-800 cm⁻¹ (C—H and C—C single bond stretch), comparable to other slippery nanoemulsion surfaces.⁴⁴ These spectral changes indicate the nanoemulsion's presence and suggest successful chemical modification, providing insight into the chemical basis for the system's functional properties.

### Surface Wettability

### Sliding Angle

A common challenge that indwelling biomaterials face is biofouling due to bacterial or blood component adhesion to the surface. The fouling material interacts with the biomaterial surface and accumulates until the device failure, resulting in complications such as invasive procedures to remove the biomaterial, prolonged hospital stays, and increased expenses.^{45,46} A key component of an effective slippery coating on biomaterials is its longevity and resilience after interacting with biological foulants, such as bacteria or blood proteins. While static water contact angle is an effective way to assess a surface's wettability, it does not necessarily demonstrate the slippery behavior of a liquid-infused surface. Instead, one metric to assess a slippery coating is measuring a surface's sliding angle, the angle at which a liquid droplet must slide down the surface. A surface is considered slippery if its sliding angle is equal to or less than 20°,⁴² indicating low surface interactions.

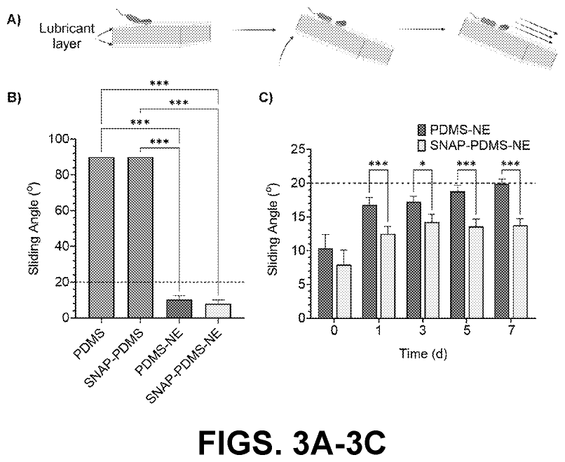

The PDMS control and SNAP-PDMS control samples pinned water to its surface without allowing for sliding when adjusting the angle at which the platform is tilted and was reported as 90°. The sliding angles of PDMS-NE and SNAP-PDMS-NE were less than that of control PDMS, indicating an increasing capability towards anti-fouling effects (FIG. 3A). The NE-swelled surfaces, expectedly, had lower sliding angles than their non-NE-swelled counterparts due to the NE's encapsulation and subsequent sliding of the fouling liquid, effectively demonstrating their slippery behavior. The NE's n-hexadecane oil base with a relatively low surface tension⁴⁷ and the high interfacial tension between the oil phase and the water droplet attributes to the ability of the water to easily slide off the slippery surface. The lubricant layer essentially created a smooth surface with which immiscible liquids or solid foulants would be able to slide off.²⁶

### Nanoemulsion Coating Stability

Implanted medical devices are intended to remain in the body for anywhere between a few hours to several months; therefore, the device's ability to remain unobstructed and viable to perform sufficiently is paramount. Slippery liquid-infused surfaces have the ability to maintain an anti-fouling nature through a replenishing lubricant reservoir swelled and stored within the polymer matrix.^{26,42} However, an initially low sliding angle does not necessarily indicate a slippery surface's overall ability to maintain slippery behavior for prolonged periods. For that, the sliding angle of PDMS-NE and SNAP-PDMS-NE was assessed over a 7-day period under agitation in physiological conditions. The conditions used would best mimic an implanted device in use over multiple days, such as short-term catheterization, showing the durability of the material. As shown in FIG. 3B, the sliding angles for PDMS-NE and SNAP-PDMS-NE were well below 20°. After 24 h submerged in PBS and shaking at 150 rpm, the sliding angle of the SNAP-PDMS-NE increased but remained below 20°. Sample manipulation and changes in phase interactions (i.e. liquid/lubricant vs air/lubricant) at each time point are probable causes for the increase in sliding angle overtime.⁴⁸ Here, the incubation media (PBS) under agitation and drying with cellulose wipes depleted the external coating and excess of the infused NE. That being said, because of the infusion of the NE into the polymer, the lubricant reservoir within the polymer matrix fully replenishes the slippery coating even after multiple days of agitation, as shown in the relatively constant sliding angle after 1 d. Similarly to previously reported SNAP-SLIPS materials, such as a SNAP-impregnated silicone oil-infused insulin cannula by Chug et al.,⁴⁹ which reports a sliding angle of less than 20° for at least 7 d, the SNAP-PDMS-NE slippery behavior is comparable to that of regular liquid infused materials. No other SNIPS materials have reported NE stability by any measurements over prolonged periods of time in the presence of physiological conditions. Despite the lubricant replenishing nature of the SNAP-PDMS-NE and PDMS-NE, negligible concentrations of the NE leached from the samples were detected in physiological and dynamic conditions. Therefore, this proof-of-concept demonstrates the potential of the SNAP-PDMS-NE material as a longer-term implanted material strategy that can resist fouling.

The significant difference between the sliding angle of PDMS-NE and that of SNAP-PDMS-NE is most likely due to the slight difference in the base polymer. Sylgard 184 PDMS was cured at 100° C. for 1 h, while SNAP-PDMS was cured at room temperature over 24 h. It is hypothesized that curing Sylgard 184 PDMS at 100° C. may induce more polymer chain crosslinking that does not allow much swelling. This hypothesis is corroborated by the reduced swelling ratio of PDMS as compared to that of SNAP-PDMS-NE, demonstrating the reduced ability of the PDMS to swell with the NE (FIG. 2B). Otherwise, the sustained slippery nature that the SNAP-PDMS-NE exhibits, in combination with the releasing NO, is indicative that the material will prevent bacteria and blood foulants.

### NO Release Kinetics

As previously described, NO is an endogenous gaseous molecule that can act as an antimicrobial, anti-platelet, and vasodilatory agent.⁵⁰⁻⁵² As a free radical gas, NO can freely diffuse through polymer matrices to treat both sides of the sample. Catalysis of RSNOs occurs via heat, light, or metal ion cleavage to release NO (FIG. 7C). The physiologically relevant levels of NO present in the endothelium of the human body produced by the endothelial cells range from 0.5-4×10⁻¹⁰ mol cm⁻² min⁻¹⁵³

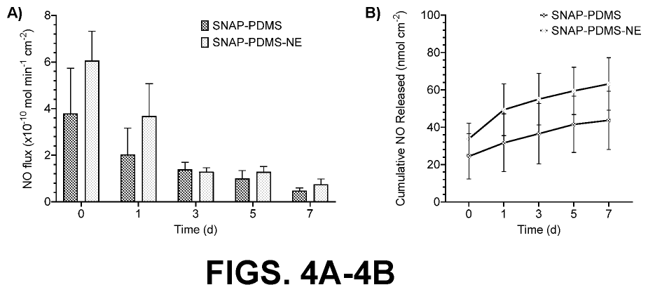

The films were weighed and measured before testing; the NO-releasing films were found to have a SNAP loading capacity of 0.0988 μmol mg⁻¹. On Day 0, both the SNAP-PDMS and SNAP-PDMS-NE films had a stabilized NO release around 3.78×10⁻¹⁰ mol cm⁻² min⁻¹ and 6.05×10⁻¹⁰ mol cm⁻² min⁻¹, respectively, but decreased to 2.03×10⁻¹⁰ mol cm⁻² min⁻¹ and 3.67×10⁻¹⁰ mol cm⁻² min⁻¹ after 24 h (FIG. 4A). The SNAP-PDMS-NE had a slightly higher release profile than SNAP-PDMS on Day 0, which can be explained by the preparation of the NE-swelled polymer. The polymer chains in the SNAP-PDMS-NE were swelled during the 4 h NE infusion to allow for the NE to form a reservoir within the polymer matrix; as the infusion time was relatively brief, there is minimized potential of significant NO loss during this time. However, in this fabrication step, the crosslinking of the SNAP-PDMS polymer chains swell enough that they would no longer be tightly linked. Therefore, the S—NO bond may be more easily broken to readily release NO.

In addition, the lubricant layer thickness is a function of the porosity of the material, with more porous polymers garnering thicker lubricant layers (between 1 and 10 μm).⁵⁴ Reports have shown that using a spin-coating technique can control the thickness of the lubricant layer;^{54,55} however, as PDMS is not a porous polymer matrix, the lubricant cannot penetrate the matrix as much as a porous membrane. Despite the lack of porosity, the SNAP-PDMS-NE sample was still thicker than the SNAP-PDMS sample due to the swelling of the polymer matrix. Not only that, as a small gaseous molecule, NO is able to penetrate through intricate polymer matrices and provide therapeutic concentrations.⁵⁶ While the loading capacity was lower than previously reported,²⁵ the NO flux on the subsequent days was more within the physiologically relevant levels^{57,58} and sustained for at least 7 days.

In previous literature, the SNAP-PDMS material has been proven to have a long-term release²⁵ due to the lack of SNAP leaching because of the covalently bound SNAP to PDMS. The covalent bond physically attaches SNAP to PDMS, which protects the NO donor molecule from undue hydrolysis that would occur in the SNAP-blended or -swelled counterparts as the SNAP molecule is only crystallized within the polymer matrix, essentially between the polymer chains, in these examples, instead of covalent attachment. Hence, the SNAP-PDMS-NE material is believed to have the longevity required for implantation applications. These previous studies with SNAP-PDMS reported a slightly higher flux profile than that of the SNAP-PDMS and SNAP-PDMS-NE in the present study in the initial days after incubation in physiological conditions; however, as the measured NO release falls within the physiological range, the infusion of the NE does not significantly affect the long-term NO release profile, thereby allowing therapeutic levels to be released.²⁵

Unlike the flux profile of the present SNAP-PDMS-NE, reports of traditional SNAP-impregnated silicone rubber with slippery modifications (i.e., silicone oil infusion) exhibit a lower flux profile at Day 0 and throughout the duration of the study, around 0.3-0.5×10⁻¹⁰ mol cm⁻² min⁻¹, which is the average release of the duration.^{38,42} The authors claim that the liquid infusion acts as a hydrophobic protective barrier around the SNAP substrate, only allowing for slow hydration into the polymer for a controlled NO release.^{38,42} While SNAP-PDMS-NE demonstrates a higher average release profile compared to the aforementioned SNAP-impregnated slippery silicone rubber, this may be due to differences in the oil composition. As the n-hexadecane used for the NE infused into SNAP-PDMS does not chemically interact with the PDMS as readily,⁵⁹ there may be less protective barrier on the sample. However, SNAP-PDMS-NE still demonstrates a prolonged NO release over 7 d with a higher cumulative NO release overall (FIG. 4B), and the higher NO release on Day 0 is advantageous to prevent the immediate onset of infection after device implantation.

### In Vitro Bacterial Adhesion Assessment

Bacterial infection is one of the prevalent causes of implanted medical device failure in terms of morbidity and mortality.^{60,61} Infections caused by skin microflora create complications such as implanted device failure due to adverse immune responses and sepsis.^{1,62} At later stages of the initial infection, microbial adhesion can mature and secrete an extracellular polymeric substance (EPS) by which the bacteria protect itself from external bactericidal stimulation, such as antibiotics,⁶³ making eradication difficult. The presence of these biofilms can cause significant chronic device infections that are costly and dangerous to remove.⁴⁶ Therefore, materials like SNAP-PDMS-NE that can help prevent infections altogether or combat existing infections are sorely needed. In the present work, SNAP-PDMS as the bulk substrate releases therapeutic levels of NO that can kill bacteria through various mechanisms, including damaging the bacteria membrane and cleaving bacterial DNA;^{64,65} the NE, while not inherently antibacterial, provides a low surface tension interface that prevents adhesion of biofouling agents like bacteria. Together, SNAP-PDMS-NE demonstrates a dual-action antibacterial mechanism with active bactericidal activity in combination with passive anti-adhesion strategies.

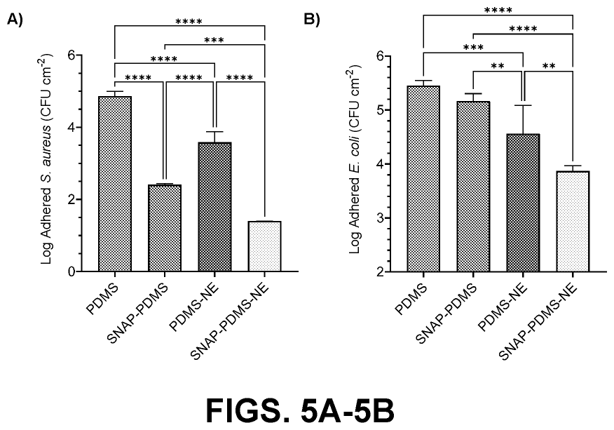

The antibacterial efficacy of the surfaces was determined against gram-positive S. aureus and gram-negative E. coli in a 24-h adhesion assay. These strains was chosen for their relevance and frequency in clinical infections⁶⁶⁻⁶⁸ to evaluate the surfaces' antibacterial activity. The SNAP-PDMS-NE samples were able to reduce bacterial adhesion of S. aureus by 3.5 log and E. coli by 1.5 log (FIGS. 5A-B) with respect to control PDMS. Significant reduction of bacterial adhesion was seen between the present SNAP-PDMS-NE, SNAP-PDMS, and PDMS-NE, demonstrating that the combination of the strategies has greater efficacy than either technique alone. As a free radical gas molecule alone, NO is able to effectively prevent bacterial proliferation through the aforementioned mechanisms.⁶⁴ The NO's ability combined with the NE's ability to prevent bacterial adhesion due to the n-hexadecane's relatively low surface tension⁴⁷ significantly reduced the number of viable bacteria adhered to the surface. The SNAP-PDMS-NE demonstrated excellent antibacterial efficacy against S. aureus while there was a slight decrease in efficacy against E. coli. However, previous reports have shown that NO's ability to reduce bacterial adhesion against gram-negative bacteria is slightly reduced due to the membrane composition of gram-negative bacteria,^{69,70} including the protective peptidoglycan cell wall and lipopolysaccharide outer membrane.⁷¹ Since S. aureus lacks the protective cell wall and outer membrane, the gram-positive strain is more susceptible to the bactericidal effects of NO. That being said, the initial concentration of the bacteria used in this experiment was higher than clinical concentrations of infections,⁷² showing that the combination SNAP-PDMS-NE surface would be especially effective against even clinical concentrations of bacteria.

### In Vitro Cell Cytocompatibility

While developing a material that can repel fouling agents and release a therapeutic agent for bactericidal activity is beneficial to maintaining the integrity of implanted medical devices, it must be ensured that the components of the material do not cause harm to the surrounding tissue. Therefore, leachates of each sample were prepared and exposed to NIH 3T3 mouse fibroblast cells to quantify the cytotoxicity of the materials fabricated.

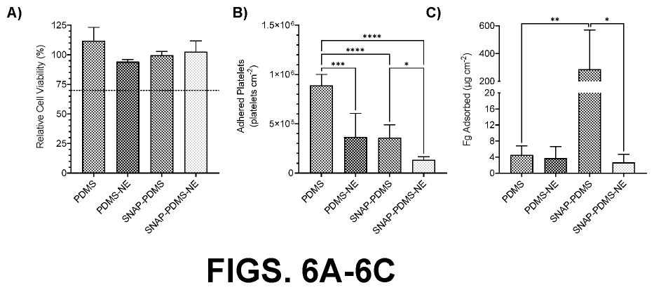

Cells exposed to the leachates from PDMS, SNAP-PDMS, PDMS-NE, and SNAP-PDMS-NE exhibited relative cell viability >70% (FIG. 6A), which falls above the ISO threshold of cell viability for medical devices.³⁶ As the n-hexadecane-based NE is the outer-most layer exposed to the cell media in the PDMS-NE and SNAP-PDMS-NE samples, it is important to note that localized irritation or tissue reaction may occur with prolonged exposure. However, due to the low toxicity of n-hexadecane^{73,74} as well as its insolubility in aqueous media,⁷⁵ the NE-infused samples were able to maintain a biocompatible relative cell viability. The cytocompatibility of the SNAP-PDMS-NE material is especially advantageous. The results indicated that all samples were biocompatible as they are above the 70% viability threshold of the ISO standard³⁶ and are viable for biological use.

### In Vitro Hemocompatibility Assessment

Thrombosis is one of the major challenges that indwelling medical devices have as an inherent bodily defense mechanism. Thrombosis is induced when a signaling cascade of clotting factors is triggered, and platelets begin to aggregate to form a platelet plug. The irreversible adhesion of blood proteins (e.g., fibrinogen) on the material's surface occurs, further fouling the surface of the device. For blood-contacting devices such as catheters or extracorporeal life support, thrombus formation can be incredibly detrimental to proper blood circulation and oxygenation; occlusion of the catheter lumen due to thrombus prevents adequate blood flow and drug administration, leaving the patient more susceptible to potential embolisms (e.g., pulmonary embolism) and high blood pressure. Traditional anticoagulant methods use systemic administration, which has adverse effects of excess bleeding, bruising, or thrombocytopenia (in the case of heparin).^{76,77}

Nitric oxide has platelet-modulating capabilities⁵² in that the endogenous gaseous NO maintains the dormant state of platelets, which prevents platelet adhesion to foreign surfaces; however, incorporating slippery modifications can further reduce the incidence of platelet adhesion and aggregation.^{42,78} In the presence of NO, platelets will be influenced to maintain innate morphology rather than be triggered by the foreign surface. Sample surface was exposed to porcine PRP for 90 min at 37° C. with subsequent quantification of adhered platelets. The PDMS-NE and SNAP-PDMS-NE exhibited a 75.68±0.011% and 89.92±0.005% reduction of adhered platelets on sample surfaces, respectively, compared to the control PDMS (FIG. 6B). The NE control's anti-platelet behavior performed similarly to that of SNAP-PDMS alone, indicating that the active and passive strategies exhibited comparable efficacy. However, as expected, the NO-releasing slippery surface was able to prevent significantly more platelets from adhering to the material surfaces compared to SNAP-PDMS alone, which is corroborated by previous reports of anti-fouling strategies in combination with NO.⁴²

Additionally, when exposed to dilute porcine whole blood, the SNAP-PDMS-NE and its constituents did not exhibit hemolytic behavior against dilute porcine whole blood (DWB). In other words, the NO released in combination with the NE infusion from the SNAP-PDMS-NE did not elicit red blood cell lysis, resulting in hemolytic indices of <2% which indicates non-hemolytic activity. The negligible SNAP and NE leaching in combination with physiological concentrations of NO released in the first 24 h is the probable explanation for the lack of red blood cell lysis.

### In Vitro Protein Adsorption Assessment

Surface interactions with proteins remains an important aspect to consider when designing blood-contacting materials. When foreign materials are introduced into the body, damage to tissue triggers the extrinsic pathway in the coagulation cascade, leading to the common pathway.⁷⁹ The conversion of fibrinogen into fibrin in the common pathway, specifically, aids in thrombus formation, which in turn allows platelets to aggregate and clot as well.⁷⁹ In addition to platelet aggregation, proteins bound to the surface have been shown to increase bacterial adhesion to the surface, promoting infection and further disruption of the biomaterial.⁸⁰ Although NO-releasing materials exhibit excellent antibacterial and anti-platelet properties, they have been shown to have increase protein adsorption on the surfaces.⁸¹ Therefore, a comprehensive material that combines NO release with anti-fouling capabilities such as slippery surfaces is imperative to combat these clinical challenges.

As a key protein in the coagulation cascade, fibrinogen (Fg) was used as the model protein for this study. After the samples were incubated with the fluorescently-labeled Fg (FITC-Fg), the fluorescence was measured and quantified. Unsurprisingly, the amount of Fg adsorbed onto SNAP-PDMS (286.12 μg cm⁻²) was significantly greater than that on PDMS (4.52 μg cm⁻²) or SNAP-PDMS-NE (2.66 μg cm⁻²) (FIG. 6C). However, when exposed to Fg, the SNAP-PDMS-NE was able to reduce adsorbed Fg by 40.97% compared to control PDMS and by 99.07% compared to SNAP-PDMS. Since fibrinogen tends to adsorb to NO-releasing materials, the results demonstrate that the combination of and NO-releasing material with an anti-fouling strategy like slippery surfaces can significantly reduce adsorbed protein and prevent an aspect of the onset of thrombosis.

### CONCLUSION

This study assessed the efficacy of a multifunctional combination nitric oxide (NO)-releasing and slippery nanoemulsion-infused porous surface (SNIPS) for its longevity, stability, and bioactivity. The SNAP-PDMS-NE surface was able to release NO at physiologically relevant concentrations for 7 days and remain slippery (characterized by a sliding angle <20°) for 7 d. Compared to other reports of NO-releasing and slippery lubricant-infused surfaces, SNAP-PDMS-NE reports negligible amounts of SNAP and lubricant leached while maintaining NO release within physiological levels for at least 7 days.^{42,49,82} Against the gram-positive strain of Staphylococcus aureus (S. aureus) and gram-negative strain of bacteria Escherichia coli (E. coli), SNAP-PDMS-NE reduced bacterial viability by 99.98 and 96%, respectively, with respect to the control PDMS, as well as significantly reduced viable bacteria adhered compared to SNAP-PDMS and PDMS-NE alone, demonstrating the combination strategy is more effective than either stand-alone technique. This work reported comparable reduction of bacterial viability as other works with different anti-fouling strategies, such as superhydrophobic surfaces, without external influence from stimuli like UV irradiation.^{83,84} Assessment of cytocompatibility determined that SNAP-PDMS-NE was not toxic to mouse fibroblast cells according to ISO standards. The SNAP-PDMS-NE also significantly reduced the number of platelets adhered to the surface by 89.9% compared to control poly(dimethylsiloxane) (PDMS), as well as significantly reduced fibrinogen adsorption by 99% compared to SNAP-PDMS, demonstrating its anti-fouling abilities against biofouling agents.

This work conceptualized and demonstrated a proof-of-concept material that incorporated an aliphatic lubricant as a nanoemulsion (NE) into a silicone rubber matrix in a facile fabrication scheme. It bridges the gap between anticoagulant and antibacterial coatings by delivering a comprehensive solution without compromising either property. The present work enables a unique avenue for antibacterial and anti-fouling capabilities in that a non-silicone-based oil is employed as the lubricant media infused into a covalently linked NO-releasing PDMS. Unlike the previously developed SLIPS materials, the combination SNAP-PDMS-NE ushers in the possibilities for different variations of slippery lubricant-infused surfaces that do not rely on similar chemical compositions to ensure lubricant retention or controlled lubricant replenishment to the surface. The stability of the material, namely the NO release and sliding angle, indicates that the SNAP-PDMS-NE has the potential and capability to maintain the necessary integrity for medical device applications. This new material would be cost- and material-effective because it would reduce the number of implanted devices that would otherwise have to be removed due to infection or blood clotting, reduce hospitalization costs, and improve therapeutic efficacy. Future studies that explore the SNAP-PDMS-NE's ability to combat biofilm activity in dynamic conditions, such as in a 7 d dripflow bioreactor model, and its ability to prevent clotting, potentially in a 4 h in vivo extracorporeal circuit (ECC) rabbit model, should be investigated to fully characterize the material's capabilities for clinical applications. Furthermore, the potential for drug loading and delivery in the slippery nanoemulsion interface should be explored to capitalize on all facets of this material. Overall, SNAP-PDMS-NE is a promising candidate for antibacterial and anti-fouling methods for future devices (e.g., catheters, extracorporeal life support, tubing, cannulas) due to its antibacterial activity, anti-fouling capabilities, and cytocompatibility.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

1) Vanepps, J. S.; Younger, J. G. Implantable Device-Related Infection. Shock 2016, 46 (6), 597-608. DOI: 10.1097/shk.0000000000000692.
(2) Bryers, J. D. Medical biofilms. Biotechnology and Bioengineering 2008, 100 (1), 1-18. DOI: 10.1002/bit.21838.
(3) Darouiche, R. O. Treatment of Infections Associated with Surgical Implants. New England Journal of Medicine 2004, 350 (14), 1422-1429. DOI: 10.1056/nejmra035415.
(4) Crnich, C. J.; Maki, D. G. The role of intravascular devices in sepsis. Current Infectious Disease Reports 2001, 3 (6), 496-506. DOI: 10.1007/s11908-001-0086-4.
(5) Ceri, H.; Olson, M. E.; Stremick, C.; Read, R. R.; Morck, D.; Buret, A. The Calgary Biofilm Device: New Technology for Rapid Determination of Antibiotic Susceptibilities of Bacterial Biofilms. Journal of Clinical Microbiology 1999, 37 (6), 1771-1776. DOI: doi:10.1128/JCM.37.6.1771-1776.1999.
(6) Stewart, P. S.; William Costerton, J. Antibiotic resistance of bacteria in biofilms. The Lancet 2001, 358 (9276), 135-138. DOI: 10.1016/s0140-6736(01)05321-1.
(7) Rashid, M. M.; Akhtar, Z.; Chowdhury, S.; Islam, M. A.; Parveen, S.; Ghosh, P. K.; Rahman, A.; Khan, Z. H.; Islam, K.; Debnath, N.; et al. Pattern of Antibiotic Use among Hospitalized Patients according to WHO Access, Watch, Reserve (AWaRe) Classification: Findings from a Point Prevalence Survey in Bangladesh. Antibiotics 2022, 11 (6), 810. DOI: 10.3390/antibiotics 11060810.
(8) Smith, D.; Murauski, J. Pulmonary Embolism in the Postanesthesia Care Unit: A Case Study. Journal of PeriAnesthesia Nursing 2017, 32 (1), 6-14. DOI: https://doi.org/10.1016/j.jopan.2015.12.016.
(9) Holdsworth, M.; Welch, S.; Borrego, M.; Spyropoulos, A.; Mahan, C. Deep-vein thrombosis: A United States cost model for a preventable and costly adverse event. Thrombosis and Haemostasis 2011, 106 (09), 405-415. DOI: 10.1160/th11-02-0132.
(10) Salzman, E. W.; Rosenberg, R. D.; Smith, M. H.; Lindon, J. N.; Favreau, L. Effect of Heparin and Heparin Fractions on Platelet Aggregation. Journal of Clinical Investigation 1980, 65 (1), 64-73. DOI: 10.1172/jci109661.
(1) Vanepps, J. S.; Younger, J. G. Implantable Device-Related Infection. Shock 2016, 46 (6), 597-608. DOI: 10.1097/shk.0000000000000692.
(2) Bryers, J. D. Medical biofilms. Biotechnology and Bioengineering 2008, 100 (1), 1-18. DOI: 10.1002/bit.21838.
(3) Darouiche, R. O. Treatment of Infections Associated with Surgical Implants. New England Journal of Medicine 2004, 350 (14), 1422-1429. DOI: 10.1056/nejmra035415.
(4) Crnich, C. J.; Maki, D. G. The role of intravascular devices in sepsis. Current Infectious Disease Reports 2001, 3 (6), 496-506. DOI: 10.1007/s11908-001-0086-4.
(5) Ceri, H.; Olson, M. E.; Stremick, C.; Read, R. R.; Morck, D.; Buret, A. The Calgary Biofilm Device: New Technology for Rapid Determination of Antibiotic Susceptibilities of Bacterial Biofilms. Journal of Clinical Microbiology 1999, 37 (6), 1771-1776. DOI: doi:10.1128/JCM.37.6.1771-1776.1999.
(6) Stewart, P. S.; William Costerton, J. Antibiotic resistance of bacteria in biofilms. The Lancet 2001, 358 (9276), 135-138. DOI: 10.1016/s0140-6736(01)05321-1.
(7) Rashid, M. M.; Akhtar, Z.; Chowdhury, S.; Islam, M. A.; Parveen, S.; Ghosh, P. K.; Rahman, A.; Khan, Z. H.; Islam, K.; Debnath, N.; et al. Pattern of Antibiotic Use among Hospitalized Patients according to WHO Access, Watch, Reserve (AWaRe) Classification: Findings from a Point Prevalence Survey in Bangladesh. Antibiotics 2022, 11 (6), 810. DOI: 10.3390/antibiotics 11060810.
(8) Smith, D.; Murauski, J. Pulmonary Embolism in the Postanesthesia Care Unit: A Case Study. Journal of PeriAnesthesia Nursing 2017, 32 (1), 6-14. DOI: https://doi.org/10.1016/j.jopan.2015.12.016.
(9) Holdsworth, M.; Welch, S.; Borrego, M.; Spyropoulos, A.; Mahan, C. Deep-vein thrombosis: A United States cost model for a preventable and costly adverse event. Thrombosis and Haemostasis 2011, 106 (09), 405-415. DOI: 10.1160/th11-02-0132.
(10) Salzman, E. W.; Rosenberg, R. D.; Smith, M. H.; Lindon, J. N.; Favreau, L. Effect of Heparin and Heparin Fractions on Platelet Aggregation. Journal of Clinical Investigation 1980, 65 (1), 64-73. DOI: 10.1172/jci109661.
(11) Rettie, A. E. The Pharmocogenomics of Warfarin: Closing in on Personalized Medicine. Molecular Interventions 2006, 6 (4), 223-227. DOI: 10.1124/mi.6.4.8.
(12) Yeh, R. W.; Jang, I.-K. Argatroban: Update. American Heart Journal 2006, 151 (6), 1131-1138. DOI: https://doi.org/10.1016/j.ahj.2005.09.002.
(13) Freedman, M. D. Oral Anticoagulants: Pharmacodynamics, Clinical Indications and Adverse Effects. The Journal of Clinical Pharmacology 1992, 32 (3), 196-209. DOI: 10.1002/j.1552-4604.1992.tb03827.x.
(14) Wysowski, D. K.; Nourjah, P.; Swartz, L. Bleeding Complications With Warfarin Use. Archives of Internal Medicine 2007, 167 (13), 1414. DOI: 10.1001/archinte.167.13.1414.
(15) Williams, D. L. H. A chemist's view of the nitric oxide story. Organic & Biomolecular Chemistry 2003, 1 (3), 441-449. DOI: 10.1039/b209748f.
(16) Fang, F. C. Perspectives series: host/pathogen interactions. Mechanisms of nitric oxide-related antimicrobial activity. J Clin Invest 1997, 99 (12), 2818-2825. DOI: 10.1172/jci119473.
(17) Wallis, J. P. Nitric oxide and blood: a review. Transfusion Medicine 2005, 15 (1), 1-11. DOI: 10.1111/j.1365-3148.2005.00542.x.
(18) Hou, Z.; Wu, Y.; Xu, C.; Reghu, S.; Shang, Z.; Chen, J.; Pranantyo, D.; Marimuth, K.; De, P. P.; Ng, O. T.; et al. Precisely Structured Nitric-Oxide-Releasing Copolymer Brush Defeats Broad-Spectrum Catheter-Associated Biofilm Infections <i>In Vivo</i>. ACS Central Science 2020, 6 (11), 2031-2045. DOI: 10.1021/acscentsci.0c00755.
(19) Brisbois, E. J.; Kim, M.; Wang, X.; Mohammed, A.; Major, T. C.; Wu, J.; Brownstein, J.; Xi, C.; Handa, H.; Bartlett, R. H.; et al. Improved Hemocompatibility of Multilumen Catheters via Nitric Oxide (NO) Release from S-Nitroso-N-acetylpenicillamine (SNAP) Composite Filled Lumen. ACS Applied Materials & Interfaces 2016, 8 (43), 29270-29279. DOI: 10.1021/acsami.6b08707.
(20) Zhang, H.; Annich, G. M.; Miskulin, J.; Osterholzer, K.; Merz, S. I.; Bartlett, R. H.; Meyerhoff, M. E. Nitric oxide releasing silicone rubbers with improved blood compatibility: preparation, characterization, and in vivo evaluation. Biomaterials 2002, 23 (6), 1485-1494. DOI: https://doi.org/10.1016/S0142-9612(01)00274-5.
(21) Feit, C. G.; Chug, M. K.; Brisbois, E. J. Development of S-Nitroso-N-Acetylpenicillamine Impregnated Medical Grade Polyvinyl Chloride for Antimicrobial Medical Device Interfaces. ACS Applied Bio Materials 2019, 2 (10), 4335-4345. DOI: 10.1021/acsabm.9b00593.
(22) Devine, R.; Goudie, M. J.; Singha, P.; Schmiedt, C.; Douglass, M.; Brisbois, E. J.; Handa, H. Mimicking the Endothelium: Dual Action Heparinized Nitric Oxide Releasing Surface. ACS Applied Materials & Interfaces 2020, 12 (18), 20158-20171. DOI: 10.1021/acsami.9b22277.
(23) Mondal, A.; Douglass, M.; Hopkins, S. P.; Singha, P.; Tran, M.; Handa, H.; Brisbois, E. J. Multifunctional S-Nitroso-N-acetylpenicillamine-Incorporated Medical-Grade Polymer with Selenium Interface for Biomedical Applications. ACS Applied Materials & Interfaces 2019, 11 (38), 34652-34662. DOI: 10.1021/acsami.9b10610.
(24) Estes Bright, L. M.; Griffin, L.; Mondal, A.; Hopkins, S.; Ozkan, E.; Handa, H. Biomimetic gasotransmitter-releasing alginate beads for biocompatible antimicrobial therapy. Journal of Colloid and Interface Science 2022, 628, 911-921. DOI: https://doi.org/10.1016/j.jcis.2022.08.113.
(25) Hopkins, S. P.; Pant, J.; Goudie, M. J.; Schmiedt, C.; Handa, H. Achieving Long-Term Biocompatible Silicone via Covalently Immobilized S-Nitroso-N-acetylpenicillamine (SNAP) That Exhibits 4 Months of Sustained Nitric Oxide Release. ACS Applied Materials & Interfaces 2018, 10 (32), 27316-27325. DOI: 10.1021/acsami.8b08647.
(26) Wong, T.-S.; Kang, S. H.; Tang, S. K. Y.; Smythe, E. J.; Hatton, B. D.; Grinthal, A.; Aizenberg, J. Bioinspired self-repairing slippery surfaces with pressure-stable omniphobicity. Nature 2011, 477 (7365), 443-447. DOI: 10.1038/nature10447.
(27) Abdulkareem, A.; Abusrafa, A. E.; Zavahir, S.; Habib, S.; Sobolčiak, P.; Lehocky, M.; Pištěková, H.; Humpolíček, P.; Popelka, A. Novel slippery liquid-infused porous surfaces (SLIPS) based on electrospun Polydimethylsiloxane/Polystyrene fibrous structures infused with natural blackseed oil. International Journal of Molecular Sciences 2022, 23 (7), 3682.
(28) Agarwal, H.; Polaske, T. J.; Senchez-Velezquez, G.; Blackwell, H. E.; Lynn, D. M. Slippery nanoemulsion-infused porous surfaces (SNIPS): anti-fouling coatings that can host and sustain the release of water-soluble agents. Chemical Communications 2021. DOI: 10.1039/d1cc04645d.
(29) Sokolov, Y. V. Nanoemulsion formation by low-energy methods: a review. Vìsnik farmacìï 2014, 0 (3(79)), 16-19. DOI: 10.24959/nphj.14.1981.
(30) Porras, M.; Solans, C.; Gonzelez, C.; Gutierrez, J. M. Properties of water-in-oil (W/O) nano-emulsions prepared by a low-energy emulsification method. Colloids and Surfaces A: Physicochemical and Engineering Aspects 2008, 324 (1-3), 181-188. DOI: 10.1016/j.colsurfa.2008.04.012.
(31) Peng, L.-C.; Liu, C.-H.; Kwan, C.-C.; Huang, K.-F. Optimization of water-in-oil nanoemulsions by mixed surfactants. Colloids and Surfaces A: Physicochemical and Engineering Aspects 2010, 370 (1-3), 136-142. DOI: 10.1016/j.colsurfa.2010.08.060.
(32) Garren, M.; Ashcraft, M.; Crowley, D.; Brisbois, E. J.; Handa, H. Derivatization of graphene oxide nanosheets with tunable nitric oxide release for antibacterial biomaterials. J Biomed Mater Res A 2023, 111 (4), 451-464. DOI: 10.1002/jbm.a.37493.
(33) Hassan, P. A.; Rana, S.; Verma, G. Making Sense of Brownian Motion: Colloid Characterization by Dynamic Light Scattering. Langmuir 2015, 31 (1), 3-12. DOI: 10.1021/la501789z.
(34) Estes Bright, L. M.; Garren, M. R. S.; Ashcraft, M.; Kumar, A.; Husain, H.; Brisbois, E. J.; Handa, H. Dual Action Nitric Oxide and Fluoride Ion-Releasing Hydrogels for Combating Dental Caries. ACS Applied Materials & Interfaces 2022, 14 (19), 21916-21930. DOI: 10.1021/acsami.2c02301.
(35) Mondal, A.; Devine, R.; Estes, L.; Manuel, J.; Singha, P.; Mancha, J.; Palmer, M.; Handa, H. Highly hydrophobic polytetrafluoroethylene particle immobilization via polydopamine anchor layer on nitric oxide releasing polymer for biomedical applications. Journal of Colloid and Interface Science 2021, 585, 716-728. DOI: 10.1016/j.jcis.2020.10.051.
(36) International Organization for Standardization. Biological evaluation of medical devices—Part 5: Tests for in vitro cytotoxicity; ISO 10993-5: 2009; 2009.
(37) International Organization for Standardization. ISO 10993-4: 2017 Biological evaluation of medical devices—Part 4: Selection of tests for interactions with blood. International Organizational for Standardization Geneve: 2017.
(38) Homeyer, K. H.; Goudie, M. J.; Singha, P.; Handa, H. Liquid-Infused Nitric-Oxide-Releasing Silicone Foley Urinary Catheters for Prevention of Catheter-Associated Urinary Tract Infections. ACS Biomaterials Science & Engineering 2019, 5 (4), 2021-2029. DOI: 10.1021/acsbiomaterials.8b01320.
(39) Wo, Y.; Li, Z.; Brisbois, E. J.; Colletta, A.; Wu, J.; Major, T. C.; Xi, C.; Bartlett, R. H.; Matzger, A. J.; Meyerhoff, M. E. Origin of Long-Term Storage Stability and Nitric Oxide Release Behavior of CarboSil Polymer Doped with S-Nitroso-N-acetyl-d-penicillamine. ACS Applied Materials & Interfaces 2015, 7 (40), 22218-22227. DOI: 10.1021/acsami.5b07501.
(40) Jaiswal, M.; Dudhe, R.; Sharma, P. K. Nanoemulsion: an advanced mode of drug delivery system. 3 Biotech 2015, 5 (2), 123-127. DOI: 10.1007/s13205-014-0214-0.
(41) Wu, H.; Ramachandran, C.; Weiner, N. D.; Roessler, B. J. Topical transport of hydrophilic compounds using water-in-oil nanoemulsions. International Journal of Pharmaceutics 2001, 220 (1-2), 63-75. DOI: 10.1016/s0378-5173(01)00671-8.
(42) Goudie, M. J.; Pant, J.; Handa, H. Liquid-infused nitric oxide-releasing (LINORel) silicone for decreased fouling, thrombosis, and infection of medical devices. Scientific Reports 2017, 7 (1). DOI: 10.1038/s41598-017-14012-9.
(43) Chug, M. K.; Sapkota, A.; Garren, M.; Brisbois, E. J. Wearable nitric oxide-releasing antibacterial insert for preventing device-associated infections. J Control Release 2024, 375, 667-680. DOI: 10.1016/j.jconrel.2024.09.027 From NLM Medline.
(44) Lynn, D. M.; Agarwal, H. Slippery liquid-infused porous surfaces that release hydrophilic and hydrophobic agents. 2023.
(45) Dadi, N. C. T.; Radochove, B.; Vargove, J.; Bujdekove, H. Impact of Healthcare-Associated Infections Connected to Medical Devices—An Update. Microorganisms 2021, 9 (11), 2332. DOI: 10.3390/microorganisms9112332.
(46) Strobel, R. M.; Leonhardt, M.; Förster, F.; Neumann, K.; Lobbes, L. A.; Seifarth, C.; Lee, L. D.; Schineis, C. H. W.; Kamphues, C.; Weixler, B.; et al. The impact of surgical site infection—a cost analysis. Langenbeck's Archives of Surgery 2022, 407 (2), 819-828. DOI: 10.1007/s00423-021-02346-y.
(47) Klein, T.; Yan, S.; Cui, J.; Magee, J. W.; Kroenlein, K.; Rausch, M. H.; Koller, T. M.; Fröba, A. P. Liquid Viscosity and Surface Tension of n-Hexane, n-Octane, n-Decane, and n-Hexadecane up to 573 K by Surface Light Scattering. Journal of Chemical & Engineering Data 2019, 64 (9), 4116-4131. DOI: 10.1021/acs.jced.9b00525.
(48) Peppou-Chapman, S.; Neto, C. Depletion of the Lubricant from Lubricant-Infused Surfaces due to an Air/Water Interface. Langmuir 2021, 37 (10), 3025-3037. DOI: 10.1021/acs.langmuir.0c02858.
(49) Chug, M. K.; Feit, C.; Brisbois, E. J. Increasing the Lifetime of Insulin Cannula with Antifouling and Nitric Oxide Releasing Properties. ACS Applied Bio Materials 2019, 2 (12), 5965-5975. DOI: 10.1021/acsabm.9b00908.
(50) Moncada, S. Nitric oxide: discovery and impact on clinical medicine. Journal of the Royal Society of Medicine 1999, 92 (4), 164-169. DOI: 10.1177/014107689909200402.
(51) Simmonds, M. J.; Detterich, J. A.; Connes, P. Nitric oxide, vasodilation and the red blood cell. Biorheology 2014, 51 (2-3), 121-134. DOI: 10.3233/bir-140653.
(52) Suchyta, D. J.; Handa, H.; Meyerhoff, M. E. A Nitric Oxide-Releasing Heparin Conjugate for Delivery of a Combined Antiplatelet/Anticoagulant Agent. Molecular Pharmaceutics 2014, 11 (2), 645-650. DOI: 10.1021/mp400501c.
(53) Vaughn, M. W.; Kuo, L.; Liao, J. C. Estimation of nitric oxide production and reactionrates in tissue by use of a mathematical model. American Journal of Physiology—Heart and Circulatory Physiology 1998, 274 (6), H2163-H2176. DOI: 10.1152/ajpheart.1998.274.6.H2163.
(54) Muschi, M.; Brudieu, B.; Teisseire, J.; Sauret, A. Drop impact dynamics on slippery liquid-infused porous surfaces: influence of oil thickness. Soft Matter 2018, 14 (7), 1100-1107. DOI: 10.1039/c7sm02026k.
(55) Vogel, N.; Belisle, R. A.; Hatton, B.; Wong, T.-S.; Aizenberg, J. Transparency and damage tolerance of patternable omniphobic lubricated surfaces based on inverse colloidal monolayers. Nature Communications 2013, 4 (1), 2176. DOI: 10.1038/ncomms3176.
(56) Ren, H.; Bull, J. L.; Meyerhoff, M. E. Transport of Nitric Oxide (NO) in Various Biomedical grade Polyurethanes: Measurements and Modeling Impact on NO Release Properties of Medical Devices. ACS Biomaterials Science & Engineering 2016, 2 (9), 1483-1492. DOI: 10.1021/acsbiomaterials.6b00215.
(57) Xu, L.-C.; Wo, Y.; Meyerhoff, M. E.; Siedlecki, C. A. Inhibition of bacterial adhesion and biofilm formation by dual functional textured and nitric oxide releasing surfaces. Acta Biomaterialia 2017, 51, 53-65. DOI: 10.1016/j.actbio.2017.01.030.
(58) Sundaram, J.; Pant, J.; Goudie, M. J.; Mani, S.; Handa, H. Antimicrobial and Physicochemical Characterization of Biodegradable, Nitric Oxide-Releasing Nanocellulose-Chitosan Packaging Membranes. Journal of Agricultural and Food Chemistry 2016, 64 (25), 5260-5266. DOI: 10.1021/acs.jafc.6b01936.
(59) Cai, Z.; Skabeev, A.; Morozova, S.; Pham, J. T. Fluid separation and network deformation in wetting of soft and swollen surfaces. Communications Materials 2021, 2 (1). DOI: 10.1038/s43246-021-00125-2.
(60) Rimondini, L.; Fini, M.; Giardino, R. The microbial infection of biomaterials: A challenge for clinicians and researchers. A short review. J Appl Biomater Biomech 2005, 3(1), 1-10.
(61) Francolini, I.; Donelli, G.; Stoodley, P. Polymer Designs to Control Biofilm Growth on Medical Devices. Reviews in Environmental Science and Bio/Technology 2003, 2 (2-4), 307-319. DOI: 10.1023/b:resb.0000040469.26208.83.
(62) Vickery, K.; Hu, H.; Jacombs, A. S.; Bradshaw, D. A.; Deva, A. K. A review of bacterial biofilms and their role in device-associated infection. Healthcare infection 2013, 18 (2), 61-66. DOI: https://doi.org/10.1071/HI12059.
(63) Mancuso, G.; Midiri, A.; Gerace, E.; Biondo, C. Bacterial Antibiotic Resistance: The Most Critical Pathogens. Pathogens 2021, 10 (10), 1310. DOI: 10.3390/pathogens10101310.
(64) Schairer, D. O.; Chouake, J. S.; Nosanchuk, J. D.; Friedman, A. J. The potential of nitric oxide releasing therapies as antimicrobial agents. Virulence 2012, 3 (3), 271-279. DOI: 10.4161/viru.20328.
(65) Mendhi, J.; Asgari, M.; Ratheesh, G.; Prasadam, I.; Yang, Y.; Xiao, Y. Dose controlled nitric oxide-based strategies for antibacterial property in biomedical devices. Applied Materials Today 2020, 19, 100562. DOI: https://doi.org/10.1016/j.apmt.2020.100562.
(66) Sharma, G.; Sharma, S.; Sharma, P.; Chandola, D.; Dang, S.; Gupta, S.; Gabrani, R. Escherichia coli biofilm: development and therapeutic strategies. Journal of Applied Microbiology 2016, 121 (2), 309-319. DOI: 10.1111/jam.13078.
(67) Weinstein, R. A.; Darouiche, R. O. Device-Associated Infections: A Macroproblem that Starts with Microadherence. Clinical Infectious Diseases 2001, 33 (9), 1567-1572. DOI: 10.1086/323130.
(68) Pietrocola, G.; Campoccia, D.; Motta, C.; Montanaro, L.; Arciola, C. R.; Speziale, P. Colonization and Infection of Indwelling Medical Devices by Staphylococcus aureus with an Emphasis on Orthopedic Implants. International Journal of Molecular Sciences 2022, 23 (11), 5958. DOI: 10.3390/ijms23115958.
(69) Friedman, A.; Blecher, K.; Sanchez, D.; Tuckman-Vernon, C.; Gialanella, P.; Friedman, J. M.; Martinez, L. R.; Nosanchuk, J. D. Susceptibility of Gram-positive and -negative bacteria to novel nitric oxide-releasing nanoparticle technology. Virulence 2011, 2 (3), 217-221. DOI: 10.4161/viru.2.3.16161.
(70) Ashcraft, M.; Garren, M.; Lautner-Csorba, O.; Pinon, V.; Wu, Y.; Crowley, D.; Hill, J.; Morales, Y.; Bartlett, R.; Brisbois, E. J.; et al. Surface Engineering for Endothelium-Mimicking Functions to Combat Infection and Thrombosis in Extracorporeal Life Support Technologies. Advanced Healthcare Materials 2024, 13 (22), 2400492. DOI: https://doi.org/10.1002/adhm.202400492.
(71) Silhavy, T. J.; Kahne, D.; Walker, S. The Bacterial Cell Envelope. Cold Spring Harbor Perspectives in Biology 2010, 2 (5), a000414-a000414. DOI: 10.1101/cshperspect.a000414.
(72) Narayana lyengar, S.; Dietvorst, J.; Ferrer-Vilanova, A.; Guirado, G.; Munoz-Berbel, X.; Russom, A. Toward Rapid Detection of Viable Bacteria in Whole Blood for Early Sepsis Diagnostics and Susceptibility Testing. ACS Sensors 2021, 6 (9), 3357-3366. DOI: 10.1021/acssensors.1c01219.
(73) Neto, W. S.; Simöes Dutra, G. V.; de Sousa Brito Neta, M.; Chaves, S. B.; Valadares, L. F.; de Souza Júnior, F. G.; Machado, F. Nanodispersions of magnetic poly(vinyl pivalate) for biomedical applications: Synthesis and in vitro evaluation of its cytotoxicity in cancer cells. Materials Today Communications 2021, 27, 102333. DOI: https://doi.org/10.1016/j.mtcomm.2021.102333.
(74) Choo, C. Y.; Chan, K. L.; Sam, T. W.; Hitotsuyanagi, Y.; Takeya, K. The cytotoxicity and chemical constituents of the hexane fraction of Typhonium flagelliforme (Araceace). Journal of Ethnopharmacology 2001, 77 (1), 129-131. DOI: https://doi.org/10.1016/S0378-8741(01)00274-4.
(75) Tsonopoulos, C. Thermodynamic analysis of the mutual solubilities of normal alkanes and water. Fluid Phase Equilibria 1999, 156 (1), 21-33. DOI: https://doi.org/10.1016/S0378-3812(99)00021-7.
(76) Alban, S. Adverse Effects of Heparin. In Heparin—A Century of Progress, Lever, R., Mulloy, B., Page, C. P. Eds.; Springer Berlin Heidelberg, 2012; pp 211-263.
(77) Freedman, M. D. Pharmacodynamics, Clinical Indications, and Adverse Effects of Heparin. The Journal of Clinical Pharmacology 1992, 32 (7), 584-596. DOI: 10.1002/j.1552-4604.1992.tb05765.x.
(78) Roberts, T. R.; Harea, G. T.; Zang, Y.; Devine, R. P.; Maffe, P.; Handa, H.; Batchinsky, A. I. A dual-action nitric oxide-releasing slippery surface for extracorporeal organ support: Dynamic in vitro hemocompatibility evaluation. Journal of Biomedical Materials Research Part B: Applied Biomaterials 2023, 111 (4), 923-932. DOI: 10.1002/jbm.b.35202.
(79) Palta, S.; Saroa, R.; Palta, A. Overview of the coagulation system. Indian J Anaesth 2014, 58 (5), 515-523. DOI: 10.4103/0019-5049.144643 From NLM.
(80) Charville, G. W.; Hetrick, E. M.; Geer, C. B.; Schoenfisch, M. H. Reduced bacterial adhesion to fibrinogen-coated substrates via nitric oxide release. Biomaterials 2008, 29 (30), 4039-4044. DOI: https://doi.org/10.1016/j.biomaterials.2008.07.005.
(81) Lantvit, S. M.; Barrett, B. J.; Reynolds, M. M. Nitric oxide releasing material adsorbs more fibrinogen. Journal of Biomedical Materials Research Part A 2013, 101 (11), 3201-3210. DOI: https://doi.org/10.1002/jbm.a.34627.
(82) Douglass, M.; Hopkins, S.; Chug, M. K.; Kim, G.; Garren, M. R.; Ashcraft, M.; Nguyen, D. T.; Tayag, N.; Handa, H.; Brisbois, E. J. Reduction in Foreign Body Response and Improved Antimicrobial Efficacy via Silicone-Oil-Infused Nitric-Oxide-Releasing Medical-Grade Cannulas. ACS Applied Materials & Interfaces 2021, 13 (44), 52425-52434. DOI: 10.1021/acsami.1c18190.
(83) Zhang, W.; Li, S.; Wei, D.; Zheng, Z.; Han, Z.; Liu, Y. Fabrication of a fluorine-free photocatalytic superhydrophobic coating and its long-lasting anticorrosion and excellent antibacterial abilities. Progress in Organic Coatings 2023, 184, 107806. DOI: https://doi.org/10.1016/j.porgcoat.2023.107806.
(84) Raeisi, M.; Kazerouni, Y.; Mohammadi, A.; Hashemi, M.; Hejazi, I.; Seyfi, J.; Khonakdar, H. A.; Davachi, S. M. Superhydrophobic cotton fabrics coated by chitosan and titanium dioxide nanoparticles with enhanced antibacterial and UV-protecting properties. International Journal of Biological Macromolecules 2021, 171, 158-165. DOI: https://doi.org/10.1016/j.ijbiomac.2020.12.220.

We claim:

1. A composition comprising
(a) a nanonemulsion comprising a surfactant and a hydrophobic solvent; and
(b) a nitric oxide releasing material comprising a (i) a polysiloxane network and (ii) a plurality of nitric oxide-donating moieties covalently bonded to the polysiloxane network.

2. The composition of claim 1, wherein the surfactant comprises a nonionic surfactant.

3. The composition of claim 1, wherein the surfactant comprises two different nonionic surfactants.

4. The composition of claim 2, wherein the nonionic surfactant comprises polyoxyethylene (20) sorbitan monolaurate, sorbitan monolaurate, polyoxyethylene 2-lauryl ether, polyoxyethylene 4-lauryl ether, polyglycerol polyricinoleate, or any combination thereof.

5. The composition of claim 1, wherein the surfactant comprises polyoxyethylene (20) sorbitan monooleate and sorbitan monooleate.

6. The composition of claim 5, wherein the weight ratio of sorbitan monooleate to polyoxyethylene (20) sorbitan monooleate is from 2:1 to 4:1.

7. The composition of claim 1, wherein the hydrophobic solvent comprises an alkane, silicone oil, sunflower oil, lemon oil, an essential oil, paraffin oil, liquid paraffin, a fluorinated oil, carnation oil, an ionic liquid, mineral oil, an edible oils, or any combination thereof.

8. The composition of claim 7, wherein the alkane is a C6 to C20 alkane.

9. The composition of claim 1, wherein the weight ratio of the hydrophobic solvent to surfactant is from 1:1 to 3:1.

10. The composition of claim 1, wherein the surfactant comprises polyoxyethylene (20) sorbitan monooleate and sorbitan monooleate and the hydrophobic solvent comprises n-hexadecane.

11. The composition of claim 10, wherein the weight ratio of sorbitan monooleate to polyoxyethylene (20) sorbitan monooleate is from 2:1 to 4:1, and the weight ratio of n-hexadecane to the sum of polyoxyethylene (20) sorbitan monooleate and sorbitan monooleate is from 1:1 to 3:1.

12. The composition of claim 1, wherein the nitric oxide-donating moiety comprises an S-nitrosothiol

13. The composition of claim 1, wherein the nitric oxide-donating moiety is a residue of S-nitroso-N-acetyl-penicillamine, S-nitroso-N-acetyl cysteine, S-nitroso-N-acetyl cysteamine, S-nitrosoglutathione, methyl S-nitrosothioglycolate, and a derivative thereof.

14. The composition of claim 1, wherein the polysiloxane network comprises a polysiloxane crosslinked with an amine-functionalized crosslinker.

15. The composition of claim 14, wherein the polysiloxane comprises a polydimethylsiloxane, a polydiethylsiloxane, a polydipropylsiloxane, or a polydiphenylsiloxane.

16. The composition of claim 1, wherein the weight ratio of the nanoemulsion to the nitric oxide releasing material is from 1:1 to 2:1.

17. The composition of claim 1, wherein the composition further comprises a bioactive agent.

18. An article comprising at least one surface, wherein the at least one surface is coated with the composition of claim 1 or one or more components are fabricated with the composition of claim 1.

19. The article of claim 18, wherein the article comprises a medical device.

20. A method of preventing bacterial growth on a surface of an article, preventing biofilm formation on a surface of an article, or preventing fibrinogen formation on a surface of an article, the method comprising applying the composition of claim 1 to the surface.
